(19) United States
(12) Patent Application Publication
(10) Pub. No.: US 2026/0100270 A1
(43) Pub. Date: Apr. 9, 2026

(54) FAST HEALTHCARE INTEROPERABILITY RESOURCES (FHIR) EXTENSION INTEGRATION

(71) Applicant: Oracle International Corporation, Redwood Shores, CA (US)

(72) Inventors: Anuj Kaushal, Noida (IN); Aditya Kulraj Kunwar, Noida (IN); Eng Keat Khor, San Jose, CA (US); Tuck Chang, Foster City, CA (US); Ravindran Sankaran, Union City, CA (US); Srimant Misra, Bengaluru (IN)

(73) Assignee: Oracle International Corporation, Redwood Shores, CA (US)

(21) Appl. No.: 19/216,545

(22) Filed: May 22, 2025

(30) Foreign Application Priority Data
Oct. 9, 2024 (IN) 202441076601

(51) Int. Cl. G16H 40/20; G16H 70/20; H04L 67/12

(52) U.S. Cl. CPC G16H 40/20; G16H 70/20; H04L 67/12

(57) ABSTRACT

Techniques are disclosed for simplifying extensions. A method comprises receiving data that comprises a standard resource that conforms to a Fast Healthcare Interoperability Resources (FHIR) standard, wherein the standard resource includes one or more standard data elements for representing and exchanging healthcare information; determining that the standard resource further includes an extension that incorporates a non-standard data element into the standard resource, wherein the non-standard data element is defined by a structure definition that is external from the data; converting the standard resource into a normalized resource that conforms with an integration model, wherein the converting includes normalizing the non-standard data element based on a schema of the integration model to generate a normalized extension element that includes an attribute that extends the standard resource, and wherein the normalized resource comprises the one or more standard data elements and the normalized extension element; and providing the normalized resource.

### CROSS-REFERENCE TO RELATED APPLICATIONS

The present application claims the benefit of and priority to India Provisional Application No. 20/244,1076601, filed Oct. 9, 2024, the entire contents of which are incorporated herein by reference for all purposes.

### BACKGROUND

Various computer systems can implement different techniques for managing healthcare information. These techniques, however, may not be compatible across computing environments. To mitigate challenges with data sharing, standards such as the Fast Healthcare Interoperability Resources (FHIR) standard have been developed to enable electronic exchanges of healthcare information and provide healthcare interoperability, customization and case of implementation.

FHIR provides a definition for common attributes but also allows for customization of attributes as some healthcare entities may use information not specified in the standard FHIR format. As an example, FHIR does not include attributes related to race, though systems based in certain countries may require attributes related to race for patient data. FHIR addresses these issues by allowing extensions to be added to the common attributes (which may be referred to herein as “standard” attributes). The extensions enable organizations to add specific data elements that are not part of the core FHIR specification without altering the standard itself, thereby maintaining interoperability. FHIR extensions, however, can add complexity to FHIR resources that make them harder to understand and implement. FHIR extensions use a uniform resource locator (URL) as the definition of the attribute. For developers who work with extended resources, interfacing with complex FHIR can be difficult.

### BRIEF SUMMARY

Techniques disclosed herein relate to normalizing FHIR extensions. Particularly, techniques are disclosed herein for normalizing FHIR extensions so that non-standard attributes can be modeled in a fashion consistent with standard FHIR attributes.

In some embodiments, a computer-implemented method includes: receiving data that comprises a standard resource that conforms to a Fast Healthcare Interoperability Resources (FHIR) standard, wherein the standard resource includes one or more standard data elements for representing and exchanging healthcare information; determining that the standard resource further includes an extension that incorporates a non-standard data element into the standard resource, wherein the non-standard data element is defined by a structure definition that is external from the data; converting the standard resource into a normalized resource that conforms with an integration model, wherein the converting includes normalizing the non-standard data element based on a schema of the integration model to generate a normalized extension element that includes an attribute that extends the standard resource, and wherein the normalized resource comprises the one or more standard data elements and the normalized extension element; and providing the normalized resource.

In some embodiments, normalizing the non-standard data element comprises replacing one or more cells of an extension array of the extension with a concrete type data structure.

In some embodiments, the computer-implemented method further includes determining a name of the attribute, from the data or the structure definition, that extends the standard resource.

In some embodiments, the computer-implemented method further includes associating an annotation with the normalized extension element, wherein the annotation indicates that the normalized extension element is associated with the extension.

In some embodiments, computer-implemented method further includes converting the normalized resource to a second standard resource, wherein the converting comprises: identifying an occurrence of the annotation associated with the normalized extension element; and converting the normalized extension element to a second non-standard extension element according to a second schema associated with the standard resource, wherein the second schema defines the standard resource and a format of the extension.

In some embodiments, providing the normalized resource comprises generating a user interface (UI) and rendering within the UI a graphical display of the normalized resource.

Some embodiments include a system that includes one or more processing systems and one or more computer-readable media storing instructions which, when executed by the one or more processing systems, cause the system to perform part or all of the operations and/or methods disclosed herein.

Some embodiments include one or more non-transitory computer-readable media storing instructions which, when executed by one or more processing systems, cause a system to perform part or all of the operations and/or methods disclosed herein.

The techniques described above and below may be implemented in a number of ways and in a number of contexts. Several example implementations and contexts are provided with reference to the following figures, as described below in more detail. However, the following implementations and contexts are but a few of many.

### BRIEF DESCRIPTION OF THE DRAWINGS

Features, embodiments, and advantages of the present disclosure are better understood when the following Detailed Description is read with reference to the accompanying drawings.

FIG. 1 is a simplified diagram of an example environment for providing a simplified FHIR extension integration, according to certain embodiments.

FIG. 2A is a simplified block diagram of an environment incorporating an integration service 202 for normalizing extensions, according to certain embodiments.

FIG. 2B depicts an example of top-level annotations, according to certain embodiments.

FIG. 2C depicts an example of a standard resource definition and a normalized resource definition, according to certain embodiments.

FIG. 3 describes a process flow for converting a standard resource into a normalized resource using an integration engine, according to some embodiments.

FIG. 4 describes a process flow for determining a structure definition indicated by a URL of an extension, according to certain embodiments.

FIG. 5 describes a process flow for converting a normalized element to a standard element, according to certain embodiments.

FIG. 6 is a block diagram illustrating one pattern for implementing a cloud infrastructure as a service system according to certain embodiments.

FIG. 7 is a block diagram illustrating another pattern for implementing a cloud infrastructure as a service system according to certain embodiments.

FIG. 8 is a block diagram illustrating another pattern for implementing a cloud infrastructure as a service system according to certain embodiments.

FIG. 9 is a block diagram illustrating another pattern for implementing a cloud infrastructure as a service system according to certain embodiments.

FIG. 10 is a block diagram illustrating an example computer system according to certain embodiments.

### DESCRIPTION

In the following description, for the purposes of explanation, specific details are set forth in order to provide a thorough understanding of certain embodiments. However, it will be apparent that various embodiments may be practiced without these specific details. The figures and description are not intended to be restrictive. The word “exemplary” is used herein to mean “serving as an example, instance, or illustration.” Any embodiment or design described herein as “exemplary” is not necessarily to be construed as preferred or advantageous over other embodiments or designs.

### INTRODUCTION

Techniques disclosed herein relate to normalizing FHIR extensions. Particularly, techniques are disclosed herein for normalizing FHIR extensions so that non-standard attributes defined by the extensions can be modeled in a fashion that is consistent with standard FHIR attributes. Using the described techniques, a user can focus on the business problems of mapping to/from fields of different resources without having to spend time dealing with the complexity of interpreting site-specific customizations defined in FHIR resources that includes one or more extensions.

FHIR is a standard developed by Health Level Seven (HL7) for exchanging healthcare information electronically. The FHIR standard is directed to healthcare interoperability, customization, and ease of implementation. FHIR extensions allow the FHIR standard to remain flexible and adaptable to various healthcare needs. Extensions enable healthcare organizations to add specific data elements that are not part of the core FHIR specification (e.g., non-standard elements) in a standard way. These extensions allow organizations to meet their specific requirements to add non-standard elements/attributes without altering the standard itself.

From an integration perspective, however, the handling of FHIR extensions poses unique and complex challenges for Integration Platform as a Service (iPaaS) vendors. Generally, a iPaaS vendor is one or more cloud services that facilitate the aggregation and synchronization of data between different applications, data sources, and/or systems. In some examples, iPaaS can provide connectors and adapters to facilitate the integration with different systems and applications. Using techniques described herein, FHIR extensions are simplified by integrating extensibility into the integration model for resources. A user can consume and produce standard FHIR specifications thereby maintaining strict conformance to standards and interoperability

According to some configurations, data is received that includes one or more standard Fast Healthcare Interoperability Resources (FHIR) resources that have one or more standard data elements for representing and exchanging healthcare information and an extension that incorporates a non-standard data element into the standard resource. As an example, a “Patient” resource type may include standard attributes such as, but not limited to a name attribute, a gender attribute, a birthdate attribute, a contact attribute, and the like. The patient resource type may also include one or more extensions that add one or more non-standard data attributes that are not part of the core FHIR specification (e.g., race, ethnicity, . . . ). According to some configurations, the extension conforms to a standard format such as:

```
“extension”: [
  {
      “url”: “http://hl7.org/fhir/us/core/StructureDefinition/us-core-race”,
    “extension”: [
    {
      “url”: “ombCategory”,
      “valueCoding”: {
        “system”: “urn:oid:2.16.840.1.113883.6.238”,
        “code”: “2106-3”,
        “display”: “White”
    }
  },
```

As can be seen in the above example, an extension to a FHIR resource provides a URL (e.g., http://hl7.org/fhir/us/core/StructureDefinition/us-core-race) that is not part of a schema or structure definition of the resource that it extends but defines the non-standard attributes. In the current example, the extension attribute relates to a “race” attribute. Further complicating the use of extensions is that extensions are defined as arrays, but each cell of the array can be of a different type. Extensions can also be nested to multiple levels.

According to some configurations, an integration engine converts a standard resource with custom attributes (e.g. a FHIR resource with one or more extensions) into a normalized resource that allows users to easily identify, and map extended types for integration. The conversion engine can normalize the extension that includes the non-standard data element based on a schema of the integration model to generate a normalized extension element that includes an attribute that extends the standard resource. After normalization, a user may easily see the different non-standard elements defined by one or more extensions without having to access an external data source. In some configurations, a graphical user interface (UI) can be used to display the standard attributes as well as the non-standard elements that are defined by one or more extensions. The normalized resource can then be easily interacted with by a user. For instance, a user may determine a value of a non-standard attribute and/or change the value of a non-standard attribute.

In some examples, the integration engine generates an integration schema that is a normalized version of the FHIR schema. Generally, the normalized schema is annotated such that both the standard elements and the non-standard elements defined by one or more extensions can be identified without having to access the external definition associated with an extension. The normalized schema can be used by the integration engine to display the non-standard attributes as part of the standard resource and abstract the underlying differences in a FHIR standard to a user. The integration engine may generate an annotated schema to identify elements within the normalized resource associated with extensions from the standard FHIR resource. The annotated schema can then be used by the integration engine to convert a normalized schema back into a standard FHIR resource with extensions. The annotations can be used by a translation engine to convert standard FHIR resources with one or more extensions to integration friendly normalized FHIR resources that comply with the integration FHIR model. The same set of annotations can also be used to restore the normalized FHIR resources back to standard FHIR resources with extensions that follow the FHIR schema.

### Environment for Simplified FHIR Extension Integration

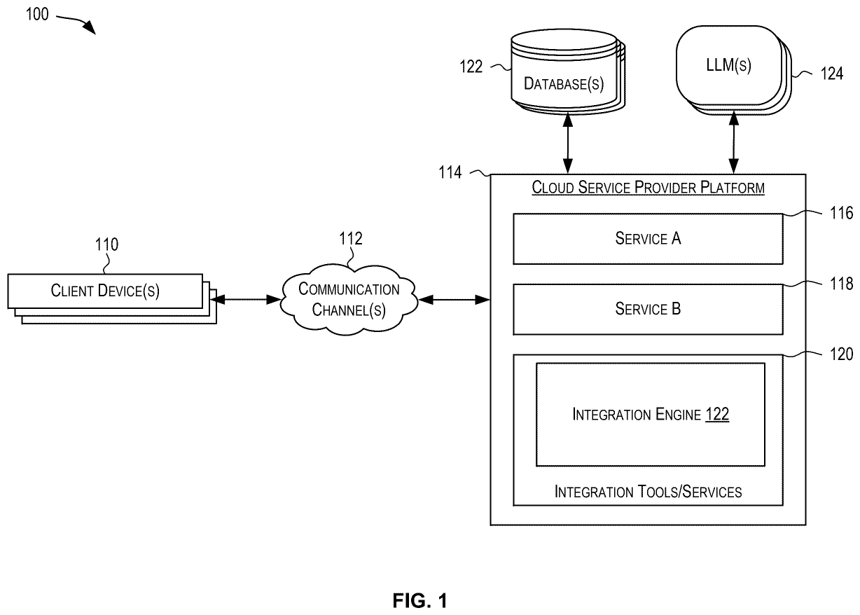

FIG. 1 is an example of an application environment 100 that includes capabilities for providing various services to various providers to facilitate management of their client populations, according to certain embodiments. In some examples, the providers may be healthcare providers and the application environment 100 may include capabilities to facilitate care and management of patient populations. The term healthcare provider generally refers to healthcare practitioners and professionals including, but not limited to: physicians (e.g., general practitioners, specialists, surgeons, etc.); nurse professionals (e.g., nurse practitioners, physician assistants, nursing staff, registered nurses, licensed practical nurses, etc.); other professionals (e.g., pharmacists, therapists, technicians, technologists, pathologists, dietitians, nutritionists, emergency medical technicians, psychiatrists, psychologists, counselors, dentists, orthodontists, hygienists, etc.).

The application environment 100 includes a cloud service provider platform 114 that includes capabilities for providing various services to subscribers (e.g., end-users) of the cloud service provider platform 114. The end-users (e.g., clinicians such as doctors and nurses) may utilize the various services provided by the cloud service provider platform 108 to perform various functions involving the treatment, care, observation, and so on of patients. For instance, in the application environment 100, the end-users can utilize the functionality provided by the services to view, edit, or manage a patient's electronic health record, perform administrative tasks such as scheduling appointments, manage patient populations, provide customer service to facilitate operation of the application environment 100, and so on.

The services provided by the cloud service provider platform 114 may include, but are not limited to, cloud integration tools and/or services 120, digital assistant services, authentication services, user management services, frontend services (e.g., entry point (façade) to all services), and other management services. The various services may be implemented on one or more servers of the cloud service provider platform 114 and may be provided to end-users who subscribe to the cloud services provided by the platform 114. In a certain implementation, the services provided by the cloud service provider platform 114 may represent digital assistant services that may be provided to enterprises or healthcare providers such as doctors, nurses, technicians, clinicians, medical personnel, and the like. For instance, the service 116 may represent an ambient service, which is an AI-powered, voice-enabled service that automatically documents patient encounters accurately and efficiently at the point of care and provides quick action suggestions. The service 118 may represent a dictation service that allows doctors to generate medical records from voice (e.g., using a Large Language Model (LLM) 124 or pre-seeded templates). As another example, the service 118 may represent a clinical automation service where healthcare providers can interact with the digital assistant, and the digital assistant can provide the end user with support for various clinical functional tasks. In some implementations, the service 120 can be integration tools to facilitate development of these services and management of other cloud services and integrations (e.g., Oracle Integration Cloud).

Various end-users may interact with the cloud service provider platform 114 using one or more client devices 110 that may be communicatively coupled to one or more servers implemented by the services (e.g., 116, 118), via one or more communication channels 112. The client devices 110 may be of various types, including but not limited to, a mobile phone, a tablet, a desktop computer, and the like. In some examples, the users can interact with the various services via a user interface (UI) of an application installed on the client devices 110 to obtain information about a patient such as medical information from an electronic health record for the patient stored in database(s) 122 (e.g. electronic health record database(s)), collect information relevant to the observation, care, treatment, and/or management of a patient, and so on.

In certain embodiments, the applications installed on the client devices 110 can support multimodal communications between an end user and the client devices 110. For example, the end user can communicate with and utilize the functionality provided by services (116, 118) via audio, voice (natural language), text, or rich user interface controls. For instance, an end user may utilize one or more voice interfaces provided by an application installed on one of the client devices 110 to interact with the services 116 and 118. In another example, the end user can interact with the application based on touch input (e.g., tapping, swiping, pinching) and voice input captured by the client device to obtain information about a patient. Voice interactions can be initiated via a wake word or by tapping a dedicated button on screen. The application can interface with the various services which can generate conversational-type responses to the voice-based interactions. In some implementations, the responses can be natural language responses and/or graphical responses. As part of a conversation with the digital assistant, an end user may provide a voice input (e.g., a natural language utterance) to one of the services 116 and 118. The platform 114 may include the capability to transcribe the voice input into text using various speech-to-text processing techniques. Components of the platform 114 may then process and determine the meaning of the text by applying natural language understanding (NLU) and/or natural language processing (NLP) techniques thereto and subsequently provide a response to the user which may be or may include a textual or audible natural language response. In one example, a user may utilize the clinical automation service to perform various clinical tasks via natural language-based conversations therewith.

In some examples, the application environment 100 additionally includes an electronic database 122. The database 12 may be a storage device managed by a healthcare provider and/or stored remotely such as in a cloud-based server or remote database managed by the cloud service provider platform 114. The database 122 may be configured to store electronic health information related to patients and/or other data such as data associated with normalizing resource data as described herein. Each electronic health record associated with a patient can be linked to other electronic health records associated with the patient. For example, one healthcare provider such as a family physician may generate an electronic health record for a patient and store that electronic health record in a local database and another healthcare provider such as a hospital may generate an electronic health record for the patient and store that electronic health record in a cloud-based database. The two electronic health records for the patient can be linked to the patient using an identifier for the patient such as a portion of the patient's personally identifiable information. While FIG. 1 shows the databases 122 and the LLMs 124 as being separate from the platform 114, this is not intended to be limiting, and one or more of the databases 122 and/or one or more of the LLMs 124 can be included as part of the platform 114 and/or the cloud infrastructure in which the platform 114 is included.

The environment 100 includes one or more integration tools/services 120. The integration tools/services 120 can be provided by a cloud-based platform (e.g. Oracle Integration Cloud (OIC)) to allow an organization such as a healthcare provider to connect applications, data, and processes across various environments, whether on-premises or in the cloud. The integration tools 120 can help integrate various applications such as services 116 and 118, databases, and third-party services. The integration tools/services 120 can be tools that allow users to create automated workflows to automate processes across integrated systems. As examples, the integrations tools/services 120 can include, but are not limited to, integrations, connections to external applications, lookups, agents, adapters, libraries, and the like. The integration tools/services 120 may also include an integration engine 122 that is configured to generate normalized resources for standard FHIR resources that include one or more extensions.

The application environment 100 depicted in FIG. 1 is merely an example and is not intended to unduly limit the scope of claimed embodiments. One of ordinary skill in the art would recognize many possible variations, alternatives, and modifications. For example, in some implementations, the application environment 100 can be implemented using more or fewer services than those shown in FIG. 1, may combine two or more services, or may have a different configuration or arrangement of services.

### Simplified FHIR Extension Generation

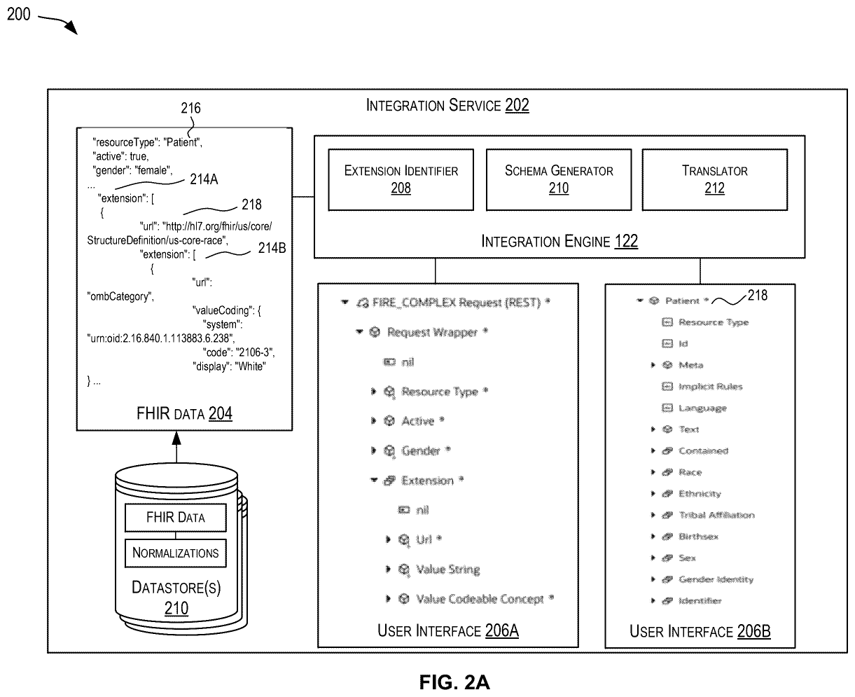

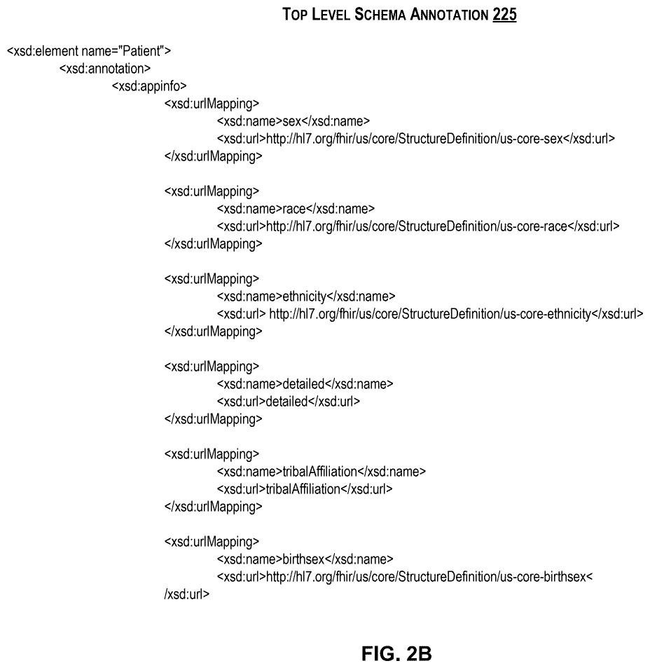

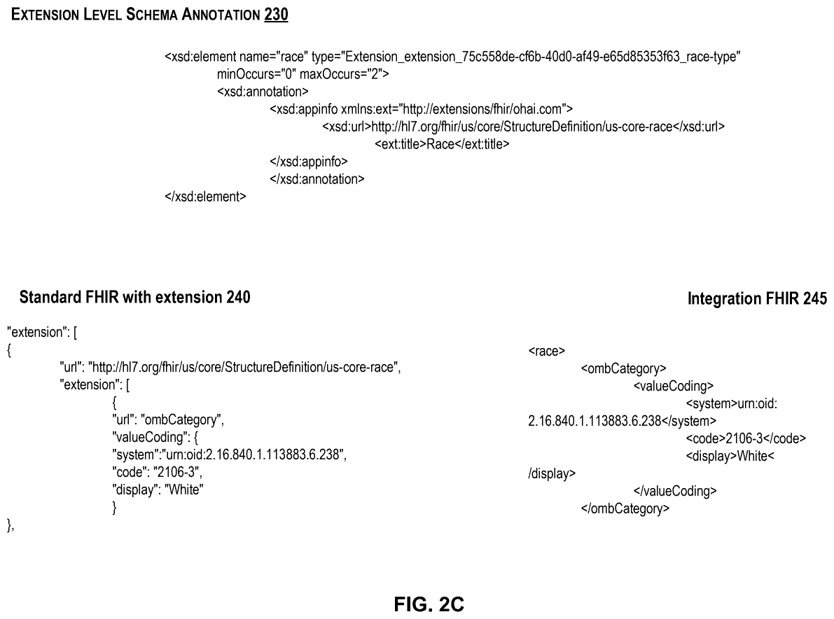

FIG. 2A is a simplified block diagram of an environment 200 incorporating an integration service 202 for normalizing extensions, according to certain embodiments. The integration service 202 includes capabilities that convert FHIR resources to normalized resources and normalized resources to FHIR resources. As illustrated, integration service 202 includes integration engine 122, user interface (UI) 206A, UI 206B, and datastore(s) 210.

According to some configurations, the integration service 202 normalizes FHIR data that includes resources and extensions so that both attributes defined by a standard FHIR resource and attributes defined by one or more extensions to the standard FHIR resource can be modeled and viewed in a fashion consistent with standard FHIR attributes. As such, a user can focus on the business problems of mapping to/from fields without having to spend time dealing with the complexity of interpreting site-specific customizations defined by one or more extensions in FHIR resources.

For purposes of illustration, FIG. 2A illustrates an example that normalizes FHIR data 204 that includes FHIR resource 216 that includes extension 214A and extension 215B such that a user can easily view both the standard attributes (e.g., the attributes defined by the FHIR resource), along with the non-standard attributes defined by the extension(s) 214.

As can be seen by referring to extension 214A within FHIR data 204, a URL that follows the “url” keyword defines the elements/attributes included within the extension. Since the definition of the non-standard attribute is defined externally from the extension a user cannot readily determine all of the attributes of the resource 216 that includes one or more extensions 214. Without normalization, a user would view the resources of the FHIR data 204 as illustrated in UI 206A. Referring to UI 206A, the user would be able to understand the standard attributes “Active” and “Gender” but would not be able to determine the non-standard attributes associated with resource 216.

After normalization is performed by integration engine 122 of integration service 202, the user can view the normalized resource 218 generated from FHIR data 204. As can be seen by referring to UI 206B, a user can easily determine the different attributes of the normalized resource 216, along with other information (e.g., name of the resource “Patient”). According to some examples, the integration service 202 can translate the normalized resource 218 back to standard resource 216.

To generate the normalized resource 218, integration engine 122 accesses FHIR data 204 that includes a resource 216 that has one or more standard attributes (e.g., active, gender, . . . ) and one or more extensions 214 that define one or more non-standard attributes (e.g., race, ethnicity) using a definition defined by the content located at a URL, such as URL 218. In some examples, extension identifier 208 is configured to parse FHIR data 204 to identify each “extension” keyword (which may be referred to as a “reserved key”) that is included within FHIR data 204. After identifying an extension by the reserved key, the URL 218 located after the “url” keyword is accessed by the integration engine 122 to determine top level annotations as illustrated in FIG. 2B. The name value corresponding to the URL is retrieved and an event is relayed for an element with this name instead. In some examples, the same process is repeated each time an “extension” is encountered within the FHIR data 204, including nested extensions. Referring to an example top level schema annotation 225 illustrated in FIG. 2B, it can be seen that the integration engine 122 has used this information to annotate each resource identified within FHIR data 204 (e.g., sex, race, ethnicity, detailed, tribal affiliation, birthsex, . . . ). In some examples, the integration engine 122 annotates each extension as indicated by extension level schema annotation 230 illustrated in FIG. 2C.

FIG. 2C also illustrates an example of a standard resource definition and a normalized resource definition 204. As discussed above, when integration engine 122 encounters the keyword “extension” in the FHIR data 204 and identifies the keyword “url”, integration engine 122 accesses the top level schema 225. For conversion back to the standard FHIR data the schema for the FHIR data element in the normalized integration FHIR resource, the annotations can be used to determine if a resource has been normalized. In some examples, if an annotation with (xmlns:ext=“http://extensions/fhir/ohai.com”) is located then that the given element is a normalized. This integration engine 122 converts the normalized element back into an extension defined according to the standard FHIR

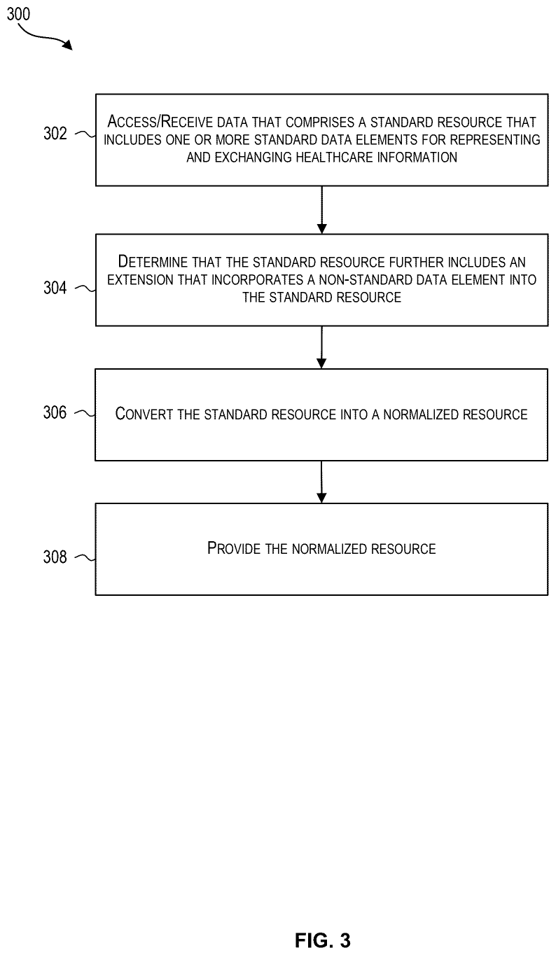

FIG. 3 depicts an example of a process 300 for converting a standard resource into a normalized resource using an integration engine 122, according to various embodiments of the present disclosure. The process depicted in FIG. 3 may be implemented in software (e.g., code, instructions, program) executed by one or more processing units (e.g., processors, cores) of the respective systems, hardware, or combinations thereof. The software may be stored on one or more non-transitory storage media (e.g., on a memory device). The process shown in FIG. 3 and described below is intended to be illustrative and non-limiting. Although FIG. 3 depicts the various steps occurring in a particular sequence or order, this is not intended to be limiting. In certain alternative embodiments, the steps may be performed in some different order or some steps may also be performed in parallel.

At 302, the process 300 can include accessing/receiving data that includes a resource. In some examples, the resource 216 is associated with a specification (e.g., FHIR) and includes a plurality of elements. The specification can include a base structure for common attributes and one or more extensions for customizable attributes. As an example, a “Patient” resource type may include attributes such as, but not limited to a name attribute, a gender attribute, a birthdate attribute, a contact attribute, and the like. The patient resource type may also include one or more extensions that add one or more non-standard data attributes that are not part of the core FHIR specification. According to some configurations, the extension follows/conforms to a standard format of

```
“extension”: [
  {
      “url”: “http://hl7.org/fhir/us/core
    /StructureDefinition/us-core-race”,
    “extension”: [
    {
      “url”: “ombCategory”,
      “valueCoding”: {
        “system”: “urn:oid:2.16.840.1.113883.6.238”,
        “code”: “2106-3”.
        “display”: “White”
    }
  },
```

As can be seen in the above example, the extension provides a URL (e.g., http://hl7.org/fhir/us/core/StructureDefinition/us-core-race) that is not part of a schema or structure definition but defines one or more non-standard attributes. In the current example, the extension attribute relates to a “race” attribute.

At 304, the process 300 can determine that the standard resource further includes one or more extensions that incorporate a non-standard data element into the standard resource. As briefly discussed above, the extension identifier 208 is configured to parse data, such as FHIR data 204, to identify “extension” keywords. After identifying the existence of the extension, the URL 218 located after the “url” keyword is accessed by the integration engine 122 to determine top level annotations as illustrated in FIG. 2B. The name value corresponding to the url is retrieved and an event is relayed for an element with this name instead. The same process is repeated each time an “extension” is encountered, including a nested extension.

At 306, the process 300 can include converting the resource into a normalized resource. As discussed above, converting the resource into a normalized resource can include for each respective element of the plurality of elements, generating a normalized element based on the respective element and a schema associated with the normalized resource. In some examples, the integration engine 122 normalizes the standard resource that includes one or more extensions to create a normalized resource that conforms with an integration model. The integration engine 122 can normalize the extension that includes the non-standard data element based on a schema of the integration model to generate a normalized extension element that includes an attribute that extends the standard resource.

After normalization, a user may easily see the different non-standard elements defined by one or more extensions without having to access an external data source. In some configurations, a graphical user interface (UI) can be used to display the names of the standard attributes as well as the non-standard elements defined by one or more extensions. The normalized resource can then be easily interacted with by a user. For instance, a user may determine a value of a non-standard attribute and/or change the value of a non-standard attribute.

As an example, an integration engine 122 can convert each standard FHIR element into an XML format, or some other format. For each identified extension within the FHIR resource, the integration engine can identify a URL associated with the extension and retrieve the attribute associated with the URL. In some examples, the integration engine 122 may retrieve the attribute from known mappings of URLs to attributes. An annotation is generated and associated with the attribute to indicate the corresponding normalized element has been converted from an FHIR extension. In some examples, the integration engine can convert the normalized resource into an FHIR resource and may convert a normalized element into an FHIR extension based on an annotation.

In some examples, the integration engine 122 generates an integration schema that is a normalized version of the FHIR schema. Generally, the normalized schema is annotated such that the non-standard elements can be easily identified. The normalized schema can display the extended attributes as part of the standard resource and abstract the underlying differences in a FHIR standard to a user. The integration model may generate an annotated schema to identify elements within the normalized resource associated with extensions from the standard FHIR resource. The annotated schema can then be used by the integration model to convert a normalized schema back into a standard FHIR resource with extensions. The annotations can be used by a translation engine to convert standard FHIR resources with one or more extensions to integration friendly normalized FHIR resources that comply with the integration FHIR model. The same set of annotations can also be used to restore the normalized FHIR resources back to standard FHIR resources with extensions that follow the FHIR schema.

At 308, the process 300 can include providing the normalized resource. In some implementations, the normalized resource can be provided to or otherwise be made available to a system, services of the services, tools of the system, and/or end users of the system. The normalized resource may contain standard elements specified in the specification and one or more custom elements associated with an extension. In some examples, the normalized resource may be displayed on a user interface of a cloud integration platform (e.g. Oracle Integration Cloud). Standard elements and customized elements may both be displayed as standard elements to allow concurrent visualization of all elements.

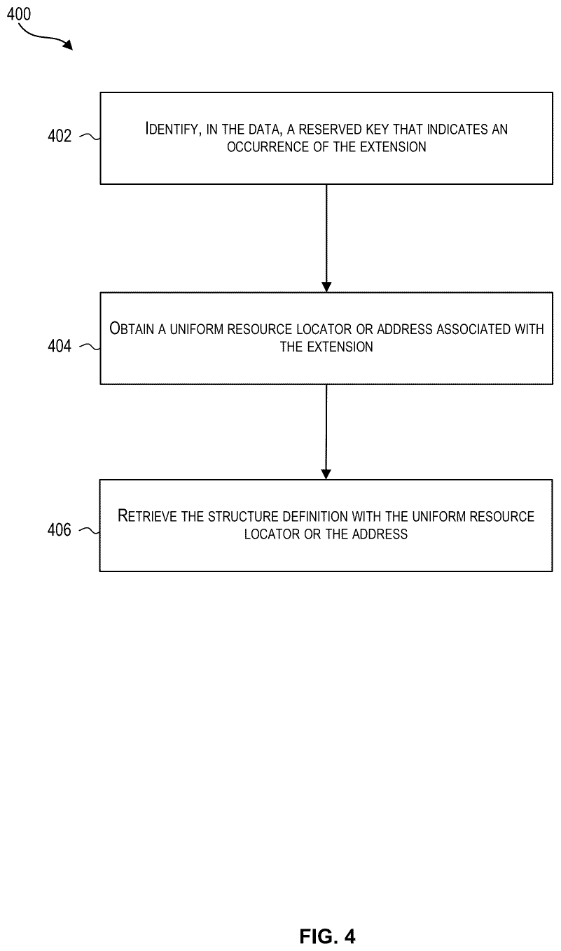

FIG. 4 depicts an example of a process 400 for determining a structure definition indicated by a URL of an extension, according to various embodiments of the present disclosure. The process depicted in FIG. 4 may be implemented in software (e.g., code, instructions, program) executed by one or more processing units (e.g., processors, cores) of the respective systems, hardware, or combinations thereof. The software may be stored on one or more non-transitory storage media (e.g., on a memory device). The process shown in FIG. 4 and described below is intended to be illustrative and non-limiting. Although FIG. 4 depicts the various steps occurring in a particular sequence or order, this is not intended to be limiting. In certain alternative embodiments, the steps may be performed in some different order or some steps may also be performed in parallel.

At 402, the process 400 can include the integration engine 122 identifying, in the data, a keyword that indicates an occurrence of the extension. In some examples, the extension identifier 208 is configured to parse FHIR data 204 and identify extensions (e.g., determine an occurrence of the “extension” keyword).

At 404, the process 400, the integration engine 122 can obtain the uniform resource locator or address associated with the extension. As discussed above, the URL can be identified by the integration engine 122 by locating the “url” keyword within the FHIR data 204.

At 406, the process 400 can include the integration engine 122 retrieving the structure definition with the uniform resource locator or the address. As discussed above, the integration engine 122 may access the location specified by the URL to identify the attribute(s) defined by the extension.

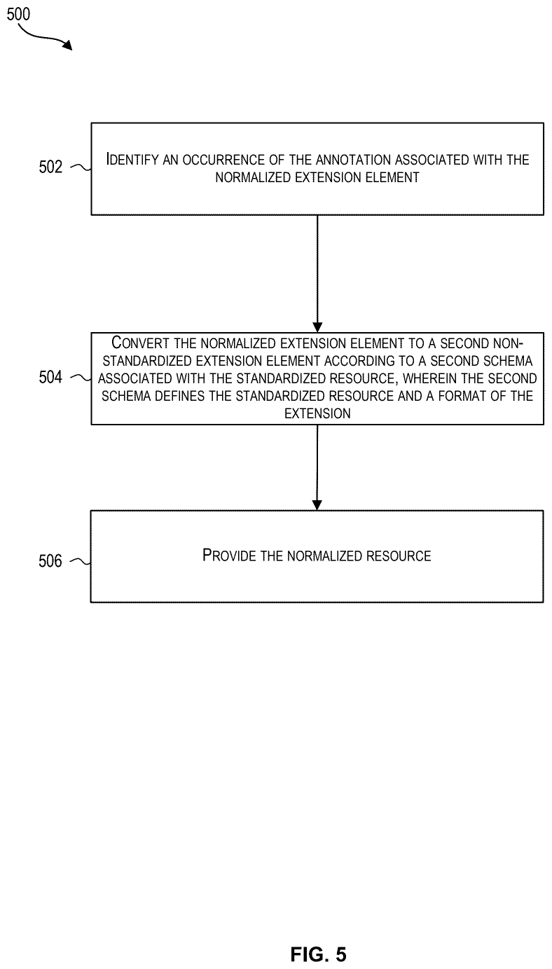

FIG. 5 depicts an example of a process 500 for converting a normalized element to a standard element, according to various embodiments of the present disclosure. The process depicted in FIG. 5 may be implemented in software (e.g., code, instructions, program) executed by one or more processing units (e.g., processors, cores) of the respective systems, hardware, or combinations thereof. The software may be stored on one or more non-transitory storage media (e.g., on a memory device). The process shown in FIG. 5 and described below is intended to be illustrative and non-limiting. Although FIG. 5 depicts the various steps occurring in a particular sequence or order, this is not intended to be limiting. In certain alternative embodiments, the steps may be performed in some different order or some steps may also be performed in parallel.

At 502, the process 500 can include the integration engine 122 identifying an occurrence of the annotation associated with the normalized extension element. In some implementations, the resource is associated with a specification and includes a plurality of elements. In some implementations, the specification associated with the resource is FHIR. The specification can include a base structure for common attributes and extensions for customizable attributes. As an example, a standard FHIR resource can include attributes associated with the standard FHIR structure (i.e. gender) and non-standard attributes (e.g. race, ethnicity) included as extensions. FHIR extension attributes are defined as values for a Uniform Resource Locator (URL) and can be included as nested arrays.

At 504, the process 500 can include the integration engine 122 converting the normalized extension element to a second non-standard extension element according to a second schema associated with the standardized resource. In some examples, the second schema defines the standardized resource and a format of the extension.

At 506, the process 500 can include the integration engine 122 providing the normalized resource. In some implementations, the normalized resource can be provided to or otherwise be made available to a system, services of the services, tools of the system, and/or end users of the system. The normalized resource may contain standard elements specified in the specification and one or more custom elements associated with an extension. In some examples, the normalized resource may be displayed on a user interface of a cloud integration platform (e.g. Oracle Integration Cloud). Standard elements and customized elements may both be displayed as standard elements to allow concurrent visualization of all elements.

### Examples Of Cloud Infrastructure Architectures

The term cloud service is generally used to refer to a service that is made available by a cloud service provider (CSP) to users (e.g., cloud service customers) on demand (e.g., via a subscription model) using systems and infrastructure (cloud infrastructure) provided by the CSP. Typically, the servers and systems that make up the CSP's infrastructure are separate from the user's own on-premise servers and systems. Users can thus avail themselves of cloud services provided by the CSP without having to purchase separate hardware and software resources for the services. Cloud services are designed to provide a subscribing user easy, scalable access to applications and computing resources without the user having to invest in procuring the infrastructure that is used for providing the services.

There are several cloud service providers that offer various types of cloud services. As discussed herein, there are various types or models of cloud services including IaaS, software as a service (SaaS), platform as a service (PaaS), and others. A user can subscribe to one or more cloud services provided by a CSP. The user can be any entity such as an individual, an organization, an enterprise, and the like. When a user subscribes to or registers for a service provided by a CSP, a tenancy or an account is created for that user. The user can then, via this account, access the subscribed-to one or more cloud resources associated with the account.

As noted above, IaaS is one particular type of cloud computing. IaaS can be configured to provide virtualized computing resources over a public network (e.g., the Internet). In an IaaS model, a cloud computing provider can host the infrastructure components (e.g., servers, storage devices, network nodes (e.g., hardware), deployment software, platform virtualization (e.g., a hypervisor layer), or the like). In some cases, an IaaS provider may also supply a variety of services to accompany those infrastructure components (example services include billing software, monitoring software, logging software, load balancing software, clustering software, etc.). Thus, as these services may be policy-driven, IaaS users may be able to implement policies to drive load balancing to maintain application availability and performance.

In some instances, IaaS customers may access resources and services through a wide area network (WAN), such as the Internet, and can use the cloud provider's services to install the remaining elements of an application stack. For example, the user can log in to the IaaS platform to create virtual machines (VMs), install operating systems (OSs) on each VM, deploy middleware such as databases, create storage buckets for workloads and backups, and even install enterprise software into that VM. Customers can then use the provider's services to perform various functions, including balancing network traffic, troubleshooting application issues, monitoring performance, managing disaster recovery, etc.

In most cases, a cloud computing model will require the participation of a cloud provider. The cloud provider may, but need not be, a third-party service that specializes in providing (e.g., offering, renting, selling) IaaS. An entity might also opt to deploy a private cloud, becoming its own provider of infrastructure services.

In some examples, IaaS deployment is the process of putting a new application, or a new version of an application, onto a prepared application server or the like. It may also include the process of preparing the server (e.g., installing libraries, daemons, etc.). This is often managed by the cloud provider, below the hypervisor layer (e.g., the servers, storage, network hardware, and virtualization). Thus, the customer may be responsible for handling (OS), middleware, and/or application deployment (e.g., on self-service virtual machines (e.g., that can be spun up on demand) or the like.

In some examples, IaaS provisioning may refer to acquiring computers or virtual hosts for use, and even installing needed libraries or services on them. In most cases, deployment does not include provisioning, and the provisioning may need to be performed first.

In some cases, there are two different challenges for IaaS provisioning. First, there is the initial challenge of provisioning the initial set of infrastructure before anything is running. Second, there is the challenge of evolving the existing infrastructure (e.g., adding new services, changing services, removing services, etc.) once everything has been provisioned. In some cases, these two challenges may be addressed by enabling the configuration of the infrastructure to be defined declaratively. In other words, the infrastructure (e.g., what components are needed and how they interact) can be defined by one or more configuration files. Thus, the overall topology of the infrastructure (e.g., what resources depend on which, and how they each work together) can be described declaratively. In some instances, once the topology is defined, a workflow can be generated that creates and/or manages the different components described in the configuration files.

In some examples, an infrastructure may have many interconnected elements. For example, there may be one or more virtual private clouds (VPCs) (e.g., a potentially on-demand pool of configurable and/or shared computing resources), also known as a core network. In some examples, there may also be one or more inbound/outbound traffic group rules provisioned to define how the inbound and/or outbound traffic of the network will be set up and one or more virtual machines (VMs). Other infrastructure elements may also be provisioned, such as a load balancer, a database, or the like. As more and more infrastructure elements are desired and/or added, the infrastructure may incrementally evolve.

In some instances, continuous deployment techniques may be employed to enable deployment of infrastructure code across various virtual computing environments. Additionally, the described techniques can enable infrastructure management within these environments. In some examples, service teams can write code that is desired to be deployed to one or more, but often many, different production environments (e.g., across various different geographic locations, sometimes spanning the entire world). However, in some examples, the infrastructure on which the code will be deployed must first be set up. In some instances, the provisioning can be done manually, a provisioning tool may be utilized to provision the resources, and/or deployment tools may be utilized to deploy the code once the infrastructure is provisioned.

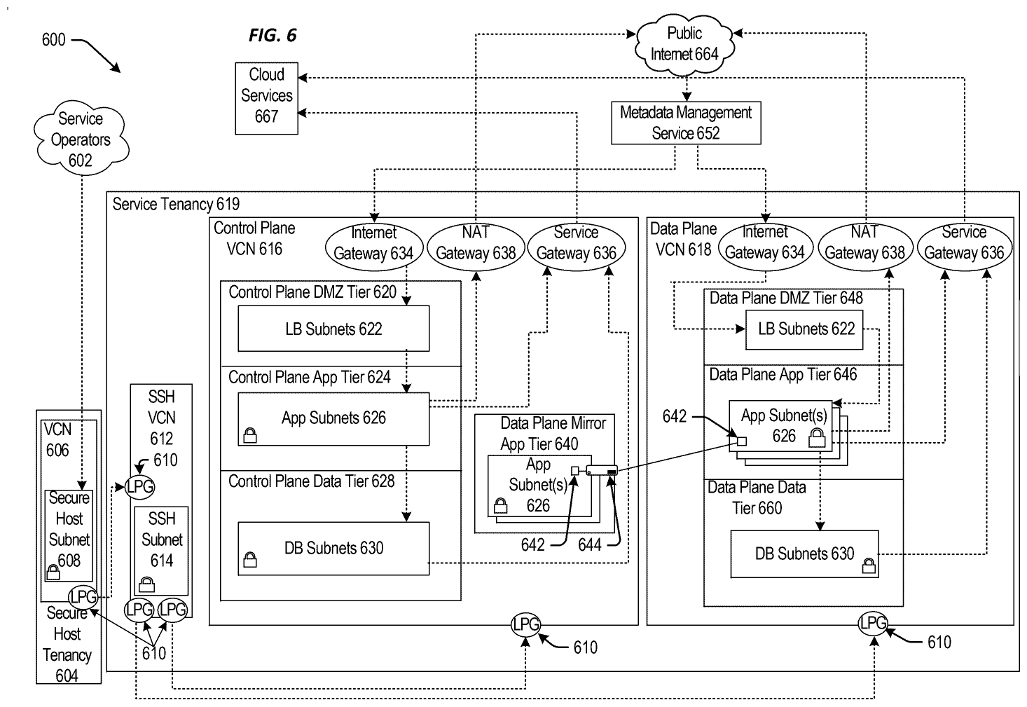

FIG. 6 is a block diagram 600 illustrating an example pattern of an IaaS architecture, according to at least one embodiment. Service operators 602 can be communicatively coupled to a secure host tenancy 604 that can include a virtual cloud network (VCN) 606 and a secure host subnet 608. In some examples, the service operators 602 may be using one or more client computing devices, which may be portable handheld devices (e.g., an iPhone®, cellular telephone, an iPad®, computing tablet, a personal digital assistant (PDA)) or wearable devices (e.g., a Google Glass® head mounted display), running software such as Microsoft Windows Mobile®, and/or a variety of mobile operating systems such as iOS, Windows Phone, Android, BlackBerry 6, Palm OS, and the like, and being Internet, e-mail, short message service (SMS), Blackberry®, or other communication protocol enabled. Alternatively, the client computing devices can be general purpose personal computers including, by way of example, personal computers and/or laptop computers running various versions of Microsoft Windows®, Apple Macintosh®, and/or Linux operating systems. The client computing devices can be workstation computers running any of a variety of commercially-available UNIX® or UNIX-like operating systems, including without limitation the variety of GNU/Linux operating systems, such as for example, Google Chrome OS. Alternatively, or in addition, client computing devices may be any other electronic device, such as a thin-client computer, an Internet-enabled gaming system (e.g., a Microsoft Xbox gaming console with or without a Kinect® gesture input device), and/or a personal messaging device, capable of communicating over a network that can access the VCN 606 and/or the Internet.

The VCN 606 can include a local peering gateway (LPG) 610 that can be communicatively coupled to a secure shell (SSH) VCN 612 via an LPG 610 contained in the SSH VCN 612. The SSH VCN 612 can include an SSH subnet 614, and the SSH VCN 612 can be communicatively coupled to a control plane VCN 616 via the LPG 610 contained in the control plane VCN 616. Also, the SSH VCN 612 can be communicatively coupled to a data plane VCN 618 via an LPG 610. The control plane VCN 616 and the data plane VCN 618 can be contained in a service tenancy 619 that can be owned and/or operated by the IaaS provider.

The control plane VCN 616 can include a control plane demilitarized zone (DMZ) tier 620 that acts as a perimeter network (e.g., portions of a corporate network between the corporate intranet and external networks). The DMZ-based servers may have restricted responsibilities and help keep breaches contained. Additionally, the control plane DMZ tier 620 can include one or more load balancer (LB) subnet(s) 622, a control plane app tier 624 that can include app subnet(s) 626, a control plane data tier 628 that can include database (DB) subnet(s) 630 (e.g., frontend DB subnet(s) and/or backend DB subnet(s)). The LB subnet(s) 622 contained in the control plane DMZ tier 620 can be communicatively coupled to the app subnet(s) 626 contained in the control plane app tier 624 and an Internet gateway 634 that can be contained in the control plane VCN 616, and the app subnet(s) 626 can be communicatively coupled to the DB subnet(s) 630 contained in the control plane data tier 628 and a service gateway 636 and a network address translation (NAT) gateway 638. The control plane VCN 616 can include the service gateway 636 and the NAT gateway 638.

The control plane VCN 616 can include a data plane mirror app tier 640 that can include app subnet(s) 626. The app subnet(s) 626 contained in the data plane mirror app tier 640 can include a virtual network interface controller (VNIC) 642 that can execute a compute instance 644. The compute instance 644 can communicatively couple the app subnet(s) 626 of the data plane mirror app tier 640 to app subnet(s) 626 that can be contained in a data plane app tier 646.

The data plane VCN 618 can include the data plane app tier 646, a data plane DMZ tier 648, and a data plane data tier 660. The data plane DMZ tier 648 can include LB subnet(s) 622 that can be communicatively coupled to the app subnet(s) 626 of the data plane app tier 646 and the Internet gateway 634 of the data plane VCN 618. The app subnet(s) 626 can be communicatively coupled to the service gateway 636 of the data plane VCN 618 and the NAT gateway 638 of the data plane VCN 618. The data plane data tier 660 can also include the DB subnet(s) 630 that can be communicatively coupled to the app subnet(s) 626 of the data plane app tier 646.

The Internet gateway 634 of the control plane VCN 616 and of the data plane VCN 618 can be communicatively coupled to a metadata management service 662 that can be communicatively coupled to public Internet 664. Public Internet 664 can be communicatively coupled to the NAT gateway 638 of the control plane VCN 616 and of the data plane VCN 618. The service gateway 636 of the control plane VCN 616 and of the data plane VCN 618 can be communicatively coupled to cloud services 667.

In some examples, the service gateway 636 of the control plane VCN 616 or of the data plane VCN 618 can make application programming interface (API) calls to cloud services 667 without going through public Internet 664. The API calls to cloud services 667 from the service gateway 636 can be one-way: the service gateway 636 can make API calls to cloud services 667, and cloud services 667 can send requested data to the service gateway 636. But, cloud services 667 may not initiate API calls to the service gateway 636.

In some examples, the secure host tenancy 604 can be directly connected to the service tenancy 619, which may be otherwise isolated. The secure host subnet 608 can communicate with the SSH subnet 614 through an LPG 610 that may enable two-way communication over an otherwise isolated system. Connecting the secure host subnet 608 to the SSH subnet 614 may give the secure host subnet 608 access to other entities within the service tenancy 619.

The control plane VCN 616 may allow users of the service tenancy 619 to set up or otherwise provision desired resources. Desired resources provisioned in the control plane VCN 616 may be deployed or otherwise used in the data plane VCN 618. In some examples, the control plane VCN 616 can be isolated from the data plane VCN 618, and the data plane mirror app tier 640 of the control plane VCN 616 can communicate with the data plane app tier 646 of the data plane VCN 618 via VNICs 642 that can be contained in the data plane mirror app tier 640 and the data plane app tier 646.

In some examples, users of the system, or customers, can make requests, for example create, read, update, or delete (CRUD) operations, through public Internet 664 that can communicate the requests to the metadata management service 662. The metadata management service 662 can communicate the request to the control plane VCN 616 through the Internet gateway 634. The request can be received by the LB subnet(s) 622 contained in the control plane DMZ tier 620. The LB subnet(s) 622 may determine that the request is valid, and in response to this determination, the LB subnet(s) 622 can transmit the request to app subnet(s) 626 contained in the control plane app tier 624. If the request is validated and requires a call to public Internet 664, the call to public Internet 664 may be transmitted to the NAT gateway 638 that can make the call to public Internet 664. Metadata that may be desired to be stored by the request can be stored in the DB subnet(s) 630.

In some examples, the data plane mirror app tier 640 can facilitate direct communication between the control plane VCN 616 and the data plane VCN 618. For example, changes, updates, or other suitable modifications to configuration may be desired to be applied to the resources contained in the data plane VCN 618. Via a VNIC 642, the control plane VCN 616 can directly communicate with, and can thereby execute the changes, updates, or other suitable modifications to configuration to, resources contained in the data plane VCN 618.

In some embodiments, the control plane VCN 616 and the data plane VCN 618 can be contained in the service tenancy 619. In this case, the user, or the customer, of the system may not own or operate either the control plane VCN 616 or the data plane VCN 618. Instead, the IaaS provider may own or operate the control plane VCN 616 and the data plane VCN 618, both of which may be contained in the service tenancy 619. This embodiment can enable isolation of networks that may prevent users or customers from interacting with other users', or other customers', resources. Also, this embodiment may allow users or customers of the system to store databases privately without needing to rely on public Internet 664, which may not have a desired level of threat prevention, for storage.

In other embodiments, the LB subnet(s) 622 contained in the control plane VCN 616 can be configured to receive a signal from the service gateway 636. In this embodiment, the control plane VCN 616 and the data plane VCN 618 may be configured to be called by a customer of the IaaS provider without calling public Internet 664. Customers of the IaaS provider may desire this embodiment since database(s) that the customers use may be controlled by the IaaS provider and may be stored on the service tenancy 619, which may be isolated from public Internet 664.

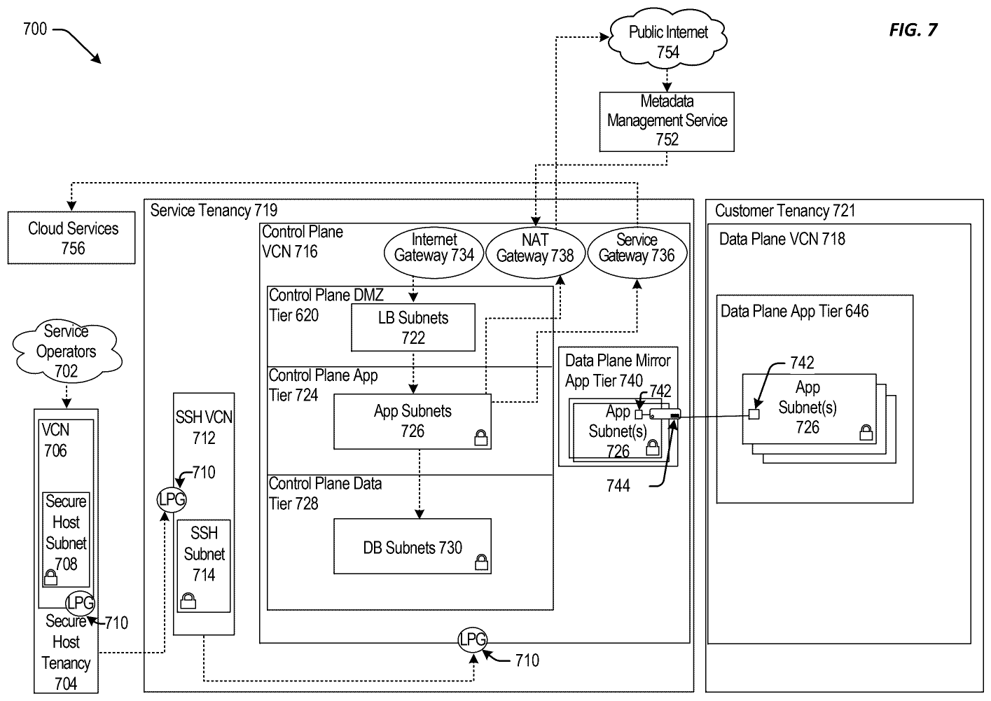

FIG. 7 is a block diagram 700 illustrating another example pattern of an IaaS architecture, according to at least one embodiment. Service operators 702 (e.g., service operators 602 of FIG. 6) can be communicatively coupled to a secure host tenancy 704 (e.g., the secure host tenancy 604 of FIG. 6) that can include a virtual cloud network (VCN) 706 (e.g., the VCN 606 of FIG. 6) and a secure host subnet 708 (e.g., the secure host subnet 608 of FIG. 6). The VCN 606 can include a local peering gateway (LPG) 710 (e.g., the LPG 610 of FIG. 6) that can be communicatively coupled to a secure shell (SSH) VCN 712 (e.g., the SSH VCN 612 of FIG. 6) via an LPG 710 contained in the SSH VCN 712. The SSH VCN 712 can include an SSH subnet 714 (e.g., the SSH subnet 614 of FIG. 6), and the SSH VCN 712 can be communicatively coupled to a control plane VCN 716 (e.g., the control plane VCN 616 of FIG. 6) via an LPG 710 contained in the control plane VCN 716. The control plane VCN 716 can be contained in a service tenancy 719 (e.g., the service tenancy 619 of FIG. 6), and the data plane VCN 718 (e.g., the data plane VCN 618 of FIG. 6) can be contained in a customer tenancy 721 that may be owned or operated by users, or customers, of the system.

The control plane VCN 716 can include a control plane DMZ tier 720 (e.g., the control plane DMZ tier 620 of FIG. 6) that can include LB subnet(s) 722 (e.g., LB subnet(s) 622 of FIG. 6), a control plane app tier 724 (e.g., the control plane app tier 624 of FIG. 6) that can include app subnet(s) 726 (e.g., app subnet(s) 626 of FIG. 6), a control plane data tier 728 (e.g., the control plane data tier 628 of FIG. 6) that can include database (DB) subnet(s) 730 (e.g., similar to DB subnet(s) 630 of FIG. 6). The LB subnet(s) 722 contained in the control plane DMZ tier 720 can be communicatively coupled to the app subnet(s) 726 contained in the control plane app tier 724 and an Internet gateway 734 (e.g., the Internet gateway 634 of FIG. 6) that can be contained in the control plane VCN 716, and the app subnet(s) 726 can be communicatively coupled to the DB subnet(s) 730 contained in the control plane data tier 728 and a service gateway 736 (e.g., the service gateway 636 of FIG. 6) and a network address translation (NAT) gateway 738 (e.g., the NAT gateway 638 of FIG. 6). The control plane VCN 716 can include the service gateway 736 and the NAT gateway 738.

The control plane VCN 716 can include a data plane mirror app tier 740 (e.g., the data plane mirror app tier 640 of FIG. 6) that can include app subnet(s) 726. The app subnet(s) 726 contained in the data plane mirror app tier 740 can include a virtual network interface controller (VNIC) 742 (e.g., the VNIC of 642) that can execute a compute instance 744 (e.g., similar to the compute instance 644 of FIG. 6). The compute instance 744 can facilitate communication between the app subnet(s) 726 of the data plane mirror app tier 740 and the app subnet(s) 726 that can be contained in a data plane app tier 746 (e.g., the data plane app tier 646 of FIG. 6) via the VNIC 742 contained in the data plane mirror app tier 740 and the VNIC 742 contained in the data plane app tier 746.

The Internet gateway 734 contained in the control plane VCN 716 can be communicatively coupled to a metadata management service 752 (e.g., the metadata management service 662 of FIG. 6) that can be communicatively coupled to public Internet 754 (e.g., public Internet 664 of FIG. 6). Public Internet 754 can be communicatively coupled to the NAT gateway 738 contained in the control plane VCN 716. The service gateway 736 contained in the control plane VCN 716 can be communicatively coupled to cloud services 756 (e.g., cloud services 667 of FIG. 6).

In some examples, the data plane VCN 718 can be contained in the customer tenancy 721. In this case, the IaaS provider may provide the control plane VCN 716 for each customer, and the IaaS provider may, for each customer, set up a unique compute instance 744 that is contained in the service tenancy 719. Each compute instance 744 may allow communication between the control plane VCN 716, contained in the service tenancy 719, and the data plane VCN 718 that is contained in the customer tenancy 721. The compute instance 744 may allow resources, that are provisioned in the control plane VCN 716 that is contained in the service tenancy 719, to be deployed or otherwise used in the data plane VCN 718 that is contained in the customer tenancy 721.

In other examples, the customer of the IaaS provider may have databases that live in the customer tenancy 721. In this example, the control plane VCN 716 can include the data plane mirror app tier 740 that can include app subnet(s) 726. The data plane mirror app tier 740 can reside in the data plane VCN 718, but the data plane mirror app tier 740 may not live in the data plane VCN 718. That is, the data plane mirror app tier 740 may have access to the customer tenancy 721, but the data plane mirror app tier 740 may not exist in the data plane VCN 718 or be owned or operated by the customer of the IaaS provider. The data plane mirror app tier 740 may be configured to make calls to the data plane VCN 718 but may not be configured to make calls to any entity contained in the control plane VCN 716. The customer may desire to deploy or otherwise use resources in the data plane VCN 718 that are provisioned in the control plane VCN 716, and the data plane mirror app tier 740 can facilitate the desired deployment, or other usage of resources, of the customer.

In some embodiments, the customer of the IaaS provider can apply filters to the data plane VCN 718. In this embodiment, the customer can determine what the data plane VCN 718 can access, and the customer may restrict access to public Internet 754 from the data plane VCN 718. The IaaS provider may not be able to apply filters or otherwise control access of the data plane VCN 718 to any outside networks or databases. Applying filters and controls by the customer onto the data plane VCN 718, contained in the customer tenancy 721, can help isolate the data plane VCN 718 from other customers and from public Internet 754.

In some embodiments, cloud services 756 can be called by the service gateway 736 to access services that may not exist on public Internet 754, on the control plane VCN 716, or on the data plane VCN 718. The connection between cloud services 756 and the control plane VCN 716 or the data plane VCN 718 may not be live or continuous. Cloud services 756 may exist on a different network owned or operated by the IaaS provider. Cloud services 756 may be configured to receive calls from the service gateway 736 and may be configured to not receive calls from public Internet 754. Some cloud services 756 may be isolated from other cloud services 756, and the control plane VCN 716 may be isolated from cloud services 756 that may not be in the same region as the control plane VCN 716. For example, the control plane VCN 716 may be located in “Region 1,” and cloud service “Deployment 8,” may be located in Region 1 and in “Region 2.” If a call to Deployment 8 is made by the service gateway 736 contained in the control plane VCN 716 located in Region 1, the call may be transmitted to Deployment 8 in Region 1. In this example, the control plane VCN 716, or Deployment 8 in Region 1, may not be communicatively coupled to, or otherwise in communication with, Deployment 8 in Region 2.

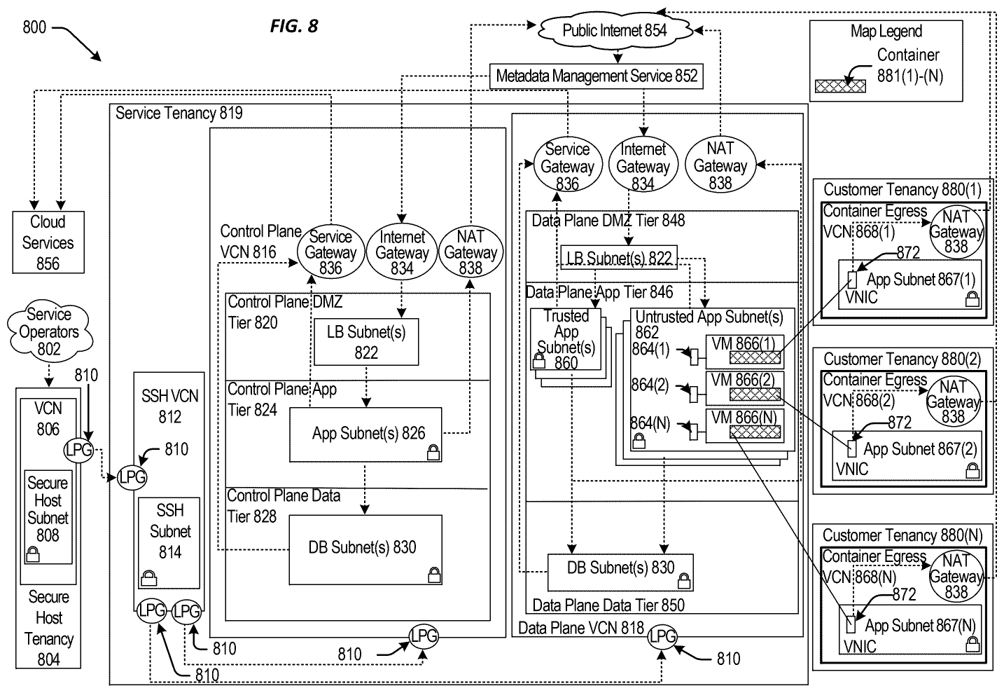

FIG. 8 is a block diagram 800 illustrating another example pattern of an IaaS architecture, according to at least one embodiment. Service operators 802 (e.g., service operators 602 of FIG. 6) can be communicatively coupled to a secure host tenancy 804 (e.g., the secure host tenancy 604 of FIG. 6) that can include a virtual cloud network (VCN) 806 (e.g., the VCN 606 of FIG. 6) and a secure host subnet 808 (e.g., the secure host subnet 608 of FIG. 6). The VCN 806 can include an LPG 810 (e.g., the LPG 610 of FIG. 6) that can be communicatively coupled to an SSH VCN 812 (e.g., the SSH VCN 612 of FIG. 6) via an LPG 810 contained in the SSH VCN 812. The SSH VCN 812 can include an SSH subnet 814 (e.g., the SSH subnet 614 of FIG. 6), and the SSH VCN 812 can be communicatively coupled to a control plane VCN 816 (e.g., the control plane VCN 616 of FIG. 6) via an LPG 810 contained in the control plane VCN 816 and to a data plane VCN 818 (e.g., the data plane VCN 618 of FIG. 6) via an LPG 810 contained in the data plane VCN 818. The control plane VCN 816 and the data plane VCN 818 can be contained in a service tenancy 819 (e.g., the service tenancy 619 of FIG. 6).

The control plane VCN 816 can include a control plane DMZ tier 820 (e.g., the control plane DMZ tier 620 of FIG. 6) that can include load balancer (LB) subnet(s) 822 (e.g., LB subnet(s) 622 of FIG. 6), a control plane app tier 824 (e.g., the control plane app tier 624 of FIG. 6) that can include app subnet(s) 826 (e.g., similar to app subnet(s) 626 of FIG. 6), a control plane data tier 828 (e.g., the control plane data tier 628 of FIG. 6) that can include DB subnet(s) 830. The LB subnet(s) 822 contained in the control plane DMZ tier 820 can be communicatively coupled to the app subnet(s) 826 contained in the control plane app tier 824 and to an Internet gateway 834 (e.g., the Internet gateway 634 of FIG. 6) that can be contained in the control plane VCN 816, and the app subnet(s) 826 can be communicatively coupled to the DB subnet(s) 830 contained in the control plane data tier 828 and to a service gateway 836 (e.g., the service gateway of FIG. 6) and a network address translation (NAT) gateway 838 (e.g., the NAT gateway 638 of FIG. 6). The control plane VCN 816 can include the service gateway 836 and the NAT gateway 838.

The data plane VCN 818 can include a data plane app tier 846 (e.g., the data plane app tier 646 of FIG. 6), a data plane DMZ tier 848 (e.g., the data plane DMZ tier 648 of FIG. 6), and a data plane data tier 850 (e.g., the data plane data tier 660 of FIG. 6). The data plane DMZ tier 848 can include LB subnet(s) 822 that can be communicatively coupled to trusted app subnet(s) 860 and untrusted app subnet(s) 862 of the data plane app tier 846 and the Internet gateway 834 contained in the data plane VCN 818. The trusted app subnet(s) 860 can be communicatively coupled to the service gateway 836 contained in the data plane VCN 818, the NAT gateway 838 contained in the data plane VCN 818, and DB subnet(s) 830 contained in the data plane data tier 850. The untrusted app subnet(s) 862 can be communicatively coupled to the service gateway 836 contained in the data plane VCN 818 and DB subnet(s) 830 contained in the data plane data tier 850. The data plane data tier 850 can include DB subnet(s) 830 that can be communicatively coupled to the service gateway 836 contained in the data plane VCN 818.

The untrusted app subnet(s) 862 can include one or more primary VNICs 864(1)-(N) that can be communicatively coupled to tenant virtual machines (VMs) 866(1)-(N). Each tenant VM 866(1)-(N) can be communicatively coupled to a respective app subnet 867(1)-(N) that can be contained in respective container egress VCNs 868(1)-(N) that can be contained in respective customer tenancies 880(1)-(N). Respective secondary VNICs 882(1)-(N) can facilitate communication between the untrusted app subnet(s) 862 contained in the data plane VCN 818 and the app subnet contained in the container egress VCNs 868(1)-(N). Each container egress VCNs 868(1)-(N) can include a NAT gateway 838 that can be communicatively coupled to public Internet 854 (e.g., public Internet 664 of FIG. 6).

The Internet gateway 834 contained in the control plane VCN 816 and contained in the data plane VCN 818 can be communicatively coupled to a metadata management service 852 (e.g., the metadata management service 662 of FIG. 6) that can be communicatively coupled to public Internet 854. Public Internet 854 can be communicatively coupled to the NAT gateway 838 contained in the control plane VCN 816 and contained in the data plane VCN 818. The service gateway 836 contained in the control plane VCN 816 and contained in the data plane VCN 818 can be communicatively coupled to cloud services 856.

In some embodiments, the data plane VCN 818 can be integrated with customer tenancies 880. This integration can be useful or desirable for customers of the IaaS provider in some cases such as a case that may desire support when executing code. The customer may provide code to run that may be destructive, may communicate with other customer resources, or may otherwise cause undesirable effects. In response to this, the IaaS provider may determine whether to run code given to the IaaS provider by the customer.

In some examples, the customer of the IaaS provider may grant temporary network access to the IaaS provider and request a function to be attached to the data plane app tier 846. Code to run the function may be executed in the VMs 866(1)-(N), and the code may not be configured to run anywhere else on the data plane VCN 818. Each VM 866(1)-(N) may be connected to one customer tenancy 880. Respective containers 881(1)-(N) contained in the VMs 866(1)-(N) may be configured to run the code. In this case, there can be a dual isolation (e.g., the containers 881(1)-(N) running code, where the containers 881(1)-(N) may be contained in at least the VM 866(1)-(N) that are contained in the untrusted app subnet(s) 862), which may help prevent incorrect or otherwise undesirable code from damaging the network of the IaaS provider or from damaging a network of a different customer. The containers 881(1)-(N) may be communicatively coupled to the customer tenancy 880 and may be configured to transmit or receive data from the customer tenancy 880. The containers 881(1)-(N) may not be configured to transmit or receive data from any other entity in the data plane VCN 818. Upon completion of running the code, the IaaS provider may kill or otherwise dispose of the containers 881(1)-(N).

In some embodiments, the trusted app subnet(s) 860 may run code that may be owned or operated by the IaaS provider. In this embodiment, the trusted app subnet(s) 860 may be communicatively coupled to the DB subnet(s) 830 and be configured to execute CRUD operations in the DB subnet(s) 830. The untrusted app subnet(s) 862 may be communicatively coupled to the DB subnet(s) 830, but in this embodiment, the untrusted app subnet(s) may be configured to execute read operations in the DB subnet(s) 830. The containers 881(1)-(N) that can be contained in the VM 866(1)-(N) of each customer and that may run code from the customer may not be communicatively coupled with the DB subnet(s) 830.

In other embodiments, the control plane VCN 816 and the data plane VCN 818 may not be directly communicatively coupled. In this embodiment, there may be no direct communication between the control plane VCN 816 and the data plane VCN 818. However, communication can occur indirectly through at least one method. An LPG 810 may be established by the IaaS provider that can facilitate communication between the control plane VCN 816 and the data plane VCN 818. In another example, the control plane VCN 816 or the data plane VCN 818 can make a call to cloud services 856 via the service gateway 836. For example, a call to cloud services 856 from the control plane VCN 816 can include a request for a service that can communicate with the data plane VCN 818.

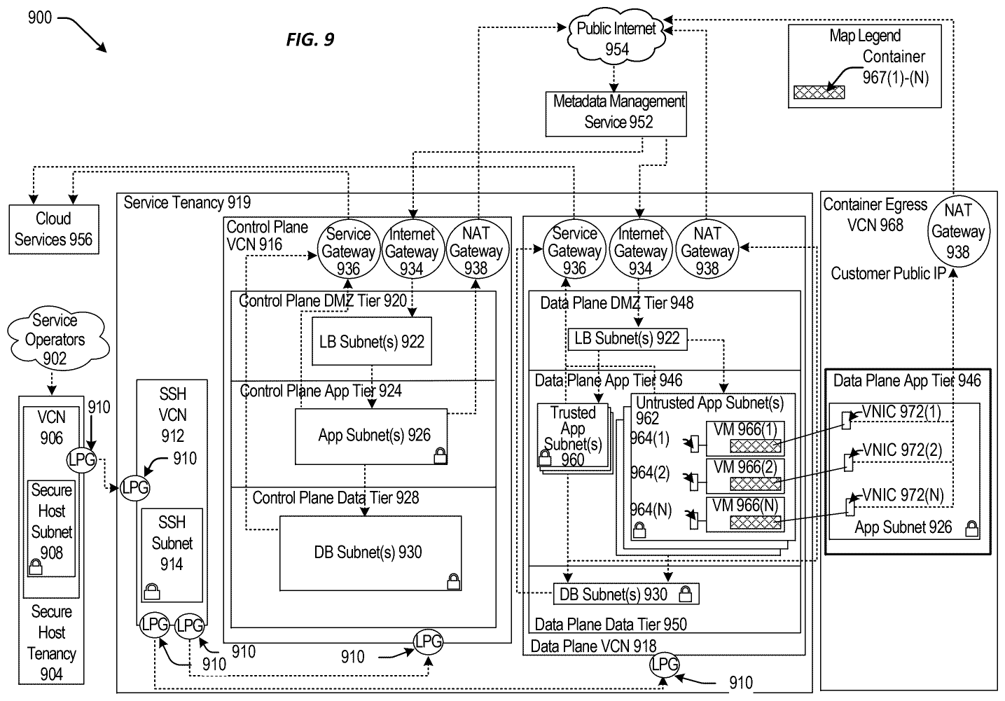

FIG. 9 is a block diagram 900 illustrating another example pattern of an IaaS architecture, according to at least one embodiment. Service operators 902 (e.g., service operators 602 of FIG. 6) can be communicatively coupled to a secure host tenancy 904 (e.g., the secure host tenancy 604 of FIG. 6) that can include a virtual cloud network (VCN) 906 (e.g., the VCN 606 of FIG. 6) and a secure host subnet 908 (e.g., the secure host subnet 608 of FIG. 6). The VCN 906 can include an LPG 910 (e.g., the LPG 610 of FIG. 6) that can be communicatively coupled to an SSH VCN 912 (e.g., the SSH VCN 612 of FIG. 6) via an LPG 910 contained in the SSH VCN 912. The SSH VCN 912 can include an SSH subnet 914 (e.g., the SSH subnet 614 of FIG. 6), and the SSH VCN 912 can be communicatively coupled to a control plane VCN 916 (e.g., the control plane VCN 616 of FIG. 6) via an LPG 910 contained in the control plane VCN 916 and to a data plane VCN 918 (e.g., the data plane VCN 618 of FIG. 6) via an LPG 910 contained in the data plane VCN 918. The control plane VCN 916 and the data plane VCN 918 can be contained in a service tenancy 919 (e.g., the service tenancy 619 of FIG. 6).

The control plane VCN 916 can include a control plane DMZ tier 920 (e.g., the control plane DMZ tier 620 of FIG. 6) that can include LB subnet(s) 922 (e.g., LB subnet(s) 622 of FIG. 6), a control plane app tier 924 (e.g., the control plane app tier 624 of FIG. 6) that can include app subnet(s) 926 (e.g., app subnet(s) 626 of FIG. 6), a control plane data tier 928 (e.g., the control plane data tier 628 of FIG. 6) that can include DB subnet(s) 930 (e.g., DB subnet(s) 730 of FIG. 7). The LB subnet(s) 922 contained in the control plane DMZ tier 920 can be communicatively coupled to the app subnet(s) 926 contained in the control plane app tier 924 and to an Internet gateway 934 (e.g., the Internet gateway 634 of FIG. 6) that can be contained in the control plane VCN 916, and the app subnet(s) 926 can be communicatively coupled to the DB subnet(s) 930 contained in the control plane data tier 928 and to a service gateway 936 (e.g., the service gateway of FIG. 6) and a network address translation (NAT) gateway 938 (e.g., the NAT gateway 638 of FIG. 6). The control plane VCN 916 can include the service gateway 936 and the NAT gateway 938.

The data plane VCN 918 can include a data plane app tier 946 (e.g., the data plane app tier 646 of FIG. 6), a data plane DMZ tier 948 (e.g., the data plane DMZ tier 648 of FIG. 6), and a data plane data tier 950 (e.g., the data plane data tier 660 of FIG. 6). The data plane DMZ tier 948 can include LB subnet(s) 922 that can be communicatively coupled to trusted app subnet(s) 960 (e.g., trusted app subnet(s) 770 of FIG. 7) and untrusted app subnet(s) 962 (e.g., untrusted app subnet(s) 772 of FIG. 7) of the data plane app tier 946 and the Internet gateway 934 contained in the data plane VCN 918. The trusted app subnet(s) 960 can be communicatively coupled to the service gateway 936 contained in the data plane VCN 918, the NAT gateway 938 contained in the data plane VCN 918, and DB subnet(s) 930 contained in the data plane data tier 950. The untrusted app subnet(s) 962 can be communicatively coupled to the service gateway 936 contained in the data plane VCN 918 and DB subnet(s) 930 contained in the data plane data tier 950. The data plane data tier 950 can include DB subnet(s) 930 that can be communicatively coupled to the service gateway 936 contained in the data plane VCN 918.

The untrusted app subnet(s) 962 can include primary VNICs 964(1)-(N) that can be communicatively coupled to tenant virtual machines (VMs) 966(1)-(N) residing within the untrusted app subnet(s) 962. Each tenant VM 966(1)-(N) can run code in a respective container 967(1)-(N), and be communicatively coupled to an app subnet 926 that can be contained in a data plane app tier 946 that can be contained in a container egress VCN 968. Respective secondary VNICs 972(1)-(N) can facilitate communication between the untrusted app subnet(s) 962 contained in the data plane VCN 918 and the app subnet contained in the container egress VCN 968. The container egress VCN can include a NAT gateway 938 that can be communicatively coupled to public Internet 954 (e.g., public Internet 664 of FIG. 6).

The Internet gateway 934 contained in the control plane VCN 916 and contained in the data plane VCN 918 can be communicatively coupled to a metadata management service 952 (e.g., the metadata management service 662 of FIG. 6) that can be communicatively coupled to public Internet 954. Public Internet 954 can be communicatively coupled to the NAT gateway 938 contained in the control plane VCN 916 and contained in the data plane VCN 918. The service gateway 936 contained in the control plane VCN 916 and contained in the data plane VCN 918 can be communicatively coupled to cloud services 956.

In some examples, the pattern illustrated by the architecture of block diagram 900 of FIG. 9 may be considered an exception to the pattern illustrated by the architecture of block diagram 700 of FIG. 7 and may be desirable for a customer of the IaaS provider if the IaaS provider cannot directly communicate with the customer (e.g., a disconnected region). The respective containers 967(1)-(N) that are contained in the VMs 966(1)-(N) for each customer can be accessed in real-time by the customer. The containers 967(1)-(N) may be configured to make calls to respective secondary VNICs 972(1)-(N) contained in app subnet(s) 926 of the data plane app tier 946 that can be contained in the container egress VCN 968. The secondary VNICs 972(1)-(N) can transmit the calls to the NAT gateway 938 that may transmit the calls to public Internet 954. In this example, the containers 967(1)-(N) that can be accessed in real-time by the customer can be isolated from the control plane VCN 916 and can be isolated from other entities contained in the data plane VCN 918. The containers 967(1)-(N) may also be isolated from resources from other customers.

In other examples, the customer can use the containers 967(1)-(N) to call cloud services 956. In this example, the customer may run code in the containers 967(1)-(N) that requests a service from cloud services 956. The containers 967(1)-(N) can transmit this request to the secondary VNICs 972(1)-(N) that can transmit the request to the NAT gateway that can transmit the request to public Internet 954. Public Internet 954 can transmit the request to LB subnet(s) 922 contained in the control plane VCN 916 via the Internet gateway 934. In response to determining the request is valid, the LB subnet(s) can transmit the request to app subnet(s) 926 that can transmit the request to cloud services 956 via the service gateway 936.

It should be appreciated that IaaS architectures 600, 700, 800, 900 depicted in the figures may have other components than those depicted. Further, the embodiments shown in the figures are only some examples of a cloud infrastructure system that may incorporate an embodiment of the disclosure. In some other embodiments, the IaaS systems may have more or fewer components than shown in the figures, may combine two or more components, or may have a different configuration or arrangement of components.

In certain embodiments, the IaaS systems described herein may include a suite of applications, middleware, and database service offerings that are delivered to a customer in a self-service, subscription-based, elastically scalable, reliable, highly available, and secure manner. An example of such an IaaS system is the Oracle Cloud Infrastructure (OCI) provided by the present assignee.

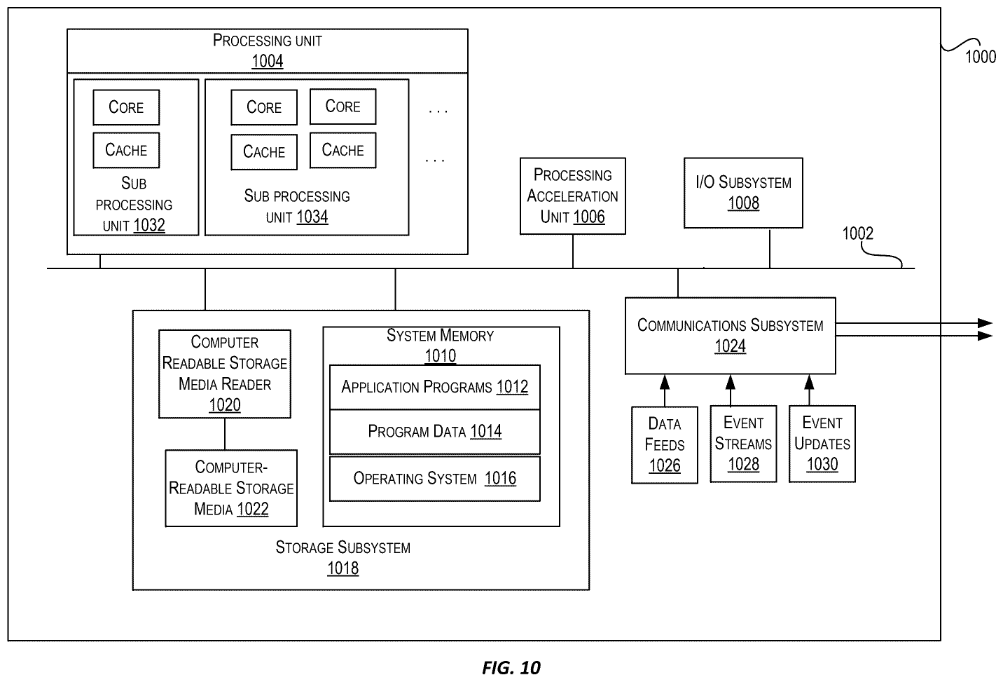

FIG. 10 illustrates an example computer system 1000, in which various embodiments may be implemented. The system 1000 may be used to implement any of the computer systems described above. As shown in the figure, computer system 1000 includes a processing unit 1004 that communicates with a number of peripheral subsystems via a bus subsystem 1002. These peripheral subsystems may include a processing acceleration unit 1006, an I/O subsystem 1008, a storage subsystem 1018 and a communications subsystem 1024. Storage subsystem 1018 includes tangible computer-readable storage media 1022 and a system memory 1010.

Bus subsystem 1002 provides a mechanism for letting the various components and subsystems of computer system 1000 communicate with each other as intended. Although bus subsystem 1002 is shown schematically as a single bus, alternative embodiments of the bus subsystem may utilize multiple buses. Bus subsystem 1002 may be any of several types of bus structures including a memory bus or memory controller, a peripheral bus, and a local bus using any of a variety of bus architectures. For example, such architectures may include an Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, which can be implemented as a Mezzanine bus manufactured to the IEEE P1386.1 standard.

Processing unit 1004, which can be implemented as one or more integrated circuits (e.g., a conventional microprocessor or microcontroller), controls the operation of computer system 1000. One or more processors may be included in processing unit 1004. These processors may include single core or multicore processors. In certain embodiments, processing unit 1004 may be implemented as one or more independent processing units 1032 and/or 1034 with single or multicore processors included in each processing unit. In other embodiments, processing unit 1004 may also be implemented as a quad-core processing unit formed by integrating two dual-core processors into a single chip.

In various embodiments, processing unit 1004 can execute a variety of programs in response to program code and can maintain multiple concurrently executing programs or processes. At any given time, some, or all of the program code to be executed can be resident in processing unit 1004 and/or in storage subsystem 1018. Through suitable programming, processing unit 1004 can provide various functionalities described above. Computer system 1000 may additionally include a processing acceleration unit 1006, which can include a digital signal processor (DSP), a special-purpose processor, and/or the like.

I/O subsystem 1008 may include user interface input devices and user interface output devices. User interface input devices may include a keyboard, pointing devices such as a mouse or trackball, a touchpad or touch screen incorporated into a display, a scroll wheel, a click wheel, a dial, a button, a switch, a keypad, audio input devices with voice command recognition systems, microphones, and other types of input devices. User interface input devices may include, for example, motion sensing and/or gesture recognition devices such as the Microsoft Kinect® motion sensor that enables users to control and interact with an input device, such as the Microsoft Xbox® 360 game controller, through a natural user interface using gestures and spoken commands. User interface input devices may also include eye gesture recognition devices such as the Google Glass® blink detector that detects eye activity (e.g., ‘blinking’ while taking pictures and/or making a menu selection) from users and transforms the eye gestures as input into an input device (e.g., Google Glass®). Additionally, user interface input devices may include voice recognition sensing devices that enable users to interact with voice recognition systems (e.g., Siri® navigator), through voice commands.

User interface input devices may also include, without limitation, three dimensional (3D) mice, joysticks or pointing sticks, gamepads and graphic tablets, and audio/visual devices such as speakers, digital cameras, digital camcorders, portable media players, webcams, image scanners, fingerprint scanners, barcode reader 3D scanners, 3D printers, laser rangefinders, and eye gaze tracking devices. Additionally, user interface input devices may include, for example, medical imaging input devices such as computed tomography, magnetic resonance imaging, position emission tomography, medical ultrasonography devices. User interface input devices may also include, for example, audio input devices such as MIDI keyboards, digital musical instruments and the like.

User interface output devices may include a display subsystem, indicator lights, or non-visual displays such as audio output devices, etc. The display subsystem may be a cathode ray tube (CRT), a flat-panel device, such as that using a liquid crystal display (LCD) or plasma display, a projection device, a touch screen, and the like. In general, use of the term “output device” is intended to include all possible types of devices and mechanisms for outputting information from computer system 1000 to a user or other computer. For example, user interface output devices may include, without limitation, a variety of display devices that visually convey text, graphics, and audio/video information such as monitors, printers, speakers, headphones, automotive navigation systems, plotters, voice output devices, and modems.

Computer system 1000 may comprise a storage subsystem 1018 that provides a tangible non-transitory computer-readable storage medium for storing software and data constructs that provide the functionality of the embodiments described in this disclosure. The software can include programs, code modules, instructions, scripts, etc., that when executed by one or more cores or processors of processing unit 1004 provide the functionality described above. Storage subsystem 1018 may also provide a repository for storing data used in accordance with the present disclosure.

As depicted in the example in FIG. 10, storage subsystem 1018 can include various components including a system memory 1010, computer-readable storage media 1022, and a computer readable storage media reader 1020. System memory 1010 may store program instructions 1012 that are loadable and executable by processing unit 1004. System memory 1010 may also store data 1014 that is used during the execution of the instructions and/or data that is generated during the execution of the program instructions. Various different kinds of programs may be loaded into system memory 1010 including but not limited to client applications, Web browsers, mid-tier applications, relational database management systems (RDBMS), virtual machines, containers, etc.

System memory 1010 may also store an operating system 1016. Examples of operating system 1016 may include various versions of Microsoft Windows®, Apple Macintosh®, and/or Linux operating systems, a variety of commercially-available UNIX® or UNIX-like operating systems (including without limitation the variety of GNU/Linux operating systems, the Google Chrome® OS, and the like) and/or mobile operating systems such as iOS, Windows® Phone, Android® OS, BlackBerry® OS, and Palm® OS operating systems. In certain implementations where computer system 1000 executes one or more virtual machines, the virtual machines along with their guest operating systems (GOSs) may be loaded into system memory 1010 and executed by one or more processors or cores of processing unit 1004.

System memory 1010 can come in different configurations depending upon the type of computer system 1000. For example, system memory 1010 may be volatile memory (such as random access memory (RAM)) and/or non-volatile memory (such as read-only memory (ROM), flash memory, etc.) Different types of RAM configurations may be provided including a static random access memory (SRAM), a dynamic random access memory (DRAM), and others. In some implementations, system memory 1010 may include a basic input/output system (BIOS) containing basic routines that help to transfer information between elements within computer system 1000, such as during start-up.

Computer-readable storage media 1022 may represent remote, local, fixed, and/or removable storage devices plus storage media for temporarily and/or more permanently containing, storing, computer-readable information for use by computer system 1000 including instructions executable by processing unit 1004 of computer system 1000.

Computer-readable storage media 1022 can include any appropriate media known or used in the art, including storage media and communication media, such as but not limited to, volatile and non-volatile, removable, and non-removable media implemented in any method or technology for storage and/or transmission of information. This can include tangible computer-readable storage media such as RAM, ROM, electronically erasable programmable ROM (EEPROM), flash memory or other memory technology, CD-ROM, digital versatile disk (DVD), or other optical storage, magnetic cassettes, magnetic tape, magnetic disk storage or other magnetic storage devices, or other tangible computer readable media.

By way of example, computer-readable storage media 1022 may include a hard disk drive that reads from or writes to non-removable, nonvolatile magnetic media, a magnetic disk drive that reads from or writes to a removable, nonvolatile magnetic disk, and an optical disk drive that reads from or writes to a removable, nonvolatile optical disk such as a CD ROM, DVD, and Blu-Ray® disk, or other optical media. Computer-readable storage media 1022 may include, but is not limited to, Zip® drives, flash memory cards, universal serial bus (USB) flash drives, secure digital (SD) cards, DVD disks, digital video tape, and the like. Computer-readable storage media 1022 may also include, solid-state drives (SSD) based on non-volatile memory such as flash-memory based SSDs, enterprise flash drives, solid state ROM, and the like, SSDs based on volatile memory such as solid state RAM, dynamic RAM, static RAM, DRAM-based SSDs, magnetoresistive RAM (MRAM) SSDs, and hybrid SSDs that use a combination of DRAM and flash memory based SSDs. The disk drives and their associated computer-readable media may provide non-volatile storage of computer-readable instructions, data structures, program modules, and other data for computer system 1000.

Machine-readable instructions executable by one or more processors or cores of processing unit 1004 may be stored on a non-transitory computer-readable storage medium. A non-transitory computer-readable storage medium can include physically tangible memory or storage devices that include volatile memory storage devices and/or non-volatile storage devices. Examples of non-transitory computer-readable storage medium include magnetic storage media (e.g., disk or tapes), optical storage media (e.g., DVDs, CDs), various types of RAM, ROM, or flash memory, hard drives, floppy drives, detachable memory drives (e.g., USB drives), or other type of storage device.

Communications subsystem 1024 provides an interface to other computer systems and networks. Communications subsystem 1024 serves as an interface for receiving data from and transmitting data to other systems from computer system 1000. For example, communications subsystem 1024 may enable computer system 1000 to connect to one or more devices via the Internet. In some embodiments communications subsystem 1024 can include radio frequency (RF) transceiver components for accessing wireless voice and/or data networks (e.g., using cellular telephone technology, advanced data network technology, such as 3G, 4G or EDGE (enhanced data rates for global evolution), WiFi (IEEE 602.10 family standards, or other mobile communication technologies, or any combination thereof), global positioning system (GPS) receiver components, and/or other components. In some embodiments communications subsystem 1024 can provide wired network connectivity (e.g., Ethernet) in addition to or instead of a wireless interface.

In some embodiments, communications subsystem 1024 may also receive input communication in the form of structured and/or unstructured data feeds 1026, event streams 1028, event updates 1030, and the like on behalf of one or more users who may use computer system 1000.

By way of example, communications subsystem 1024 may be configured to receive data feeds 1026 in real-time from users of social networks and/or other communication services such as Twitter® feeds, Facebook® updates, web feeds such as Rich Site Summary (RSS) feeds, and/or real-time updates from one or more third party information sources.

Additionally, communications subsystem 1024 may also be configured to receive data in the form of continuous data streams, which may include event streams 1028 of real-time events and/or event updates 1030, that may be continuous or unbounded in nature with no explicit end. Examples of applications that generate continuous data may include, for example, sensor data applications, financial tickers, network performance measuring tools (e.g., network monitoring and traffic management applications), clickstream analysis tools, automobile traffic monitoring, and the like.

Communications subsystem 1024 may also be configured to output the structured and/or unstructured data feeds 1026, event streams 1028, event updates 1030, and the like to one or more databases that may be in communication with one or more streaming data source computers coupled to computer system 1000.

Computer system 1000 can be one of various types, including a handheld portable device (e.g., an iPhone® cellular phone, an iPad® computing tablet, a PDA), a wearable device (e.g., a Google Glass® head mounted display), a PC, a workstation, a mainframe, a kiosk, a server rack, or any other data processing system.

Due to the ever-changing nature of computers and networks, the description of computer system 1000 depicted in the figure is intended only as a specific example. Many other configurations having more or fewer components than the system depicted in the figure are possible. For example, customized hardware might also be used and/or particular elements might be implemented in hardware, firmware, software (including applets), or a combination. Further, connection to other computing devices, such as network input/output devices, may be employed. Based on the disclosure and teachings provided herein, a person of ordinary skill in the art will appreciate other ways and/or methods to implement the various embodiments.

Although specific embodiments have been described, various modifications, alterations, alternative constructions, and equivalents are also encompassed within the scope of the disclosure. Embodiments are not restricted to operation within certain specific data processing environments but are free to operate within a plurality of data processing environments. Additionally, although embodiments have been described using a particular series of transactions and steps, it should be apparent to those skilled in the art that the scope of the present disclosure is not limited to the described series of transactions and steps. Various features and aspects of the above-described embodiments may be used individually or jointly.

Further, while embodiments have been described using a particular combination of hardware and software, it should be recognized that other combinations of hardware and software are also within the scope of the present disclosure. Embodiments may be implemented only in hardware, or only in software, or using combinations thereof. The various processes described herein can be implemented on the same processor or different processors in any combination. Accordingly, where components or services are described as being configured to perform certain operations, such configuration can be accomplished, e.g., by designing electronic circuits to perform the operation, by programming programmable electronic circuits (such as microprocessors) to perform the operation, or any combination thereof. Processes can communicate using a variety of techniques including but not limited to conventional techniques for inter process communication, and different pairs of processes may use different techniques, or the same pair of processes may use different techniques at different times.

The specification and drawings are, accordingly, to be regarded in an illustrative rather than a restrictive sense. It will, however, be evident that additions, subtractions, deletions, and other modifications and changes may be made thereunto without departing from the broader spirit and scope as set forth in the claims. Thus, although specific disclosure embodiments have been described, these are not intended to be limiting. Various modifications and equivalents are within the scope of the following claims.

The use of the terms “a” and “an” and “the” and similar referents in the context of describing the disclosed embodiments (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments and does not pose a limitation on the scope of the disclosure unless otherwise claimed. No language in the specification should be construed as indicating any non-claimed element as essential to the practice of the disclosure.

As used herein, when an action is “based on” something, this means the action is based at least in part on at least a part of the something. As used herein, the terms “substantially,” “approximately” and “about” are defined as being largely but not necessarily wholly what is specified (and include wholly what is specified) as understood by one of ordinary skill in the art. In any disclosed embodiment, the term “substantially,” “approximately,” or “about” may be substituted with “within [a percentage] of” what is specified, where the percentage includes 0.1, 1, 6, and 8 percent.

Disjunctive language such as the phrase “at least one of X, Y, or Z,” unless specifically stated otherwise, is intended to be understood within the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Preferred embodiments of this disclosure are described herein, including the best mode known for carrying out the disclosure. Variations of those preferred embodiments may become apparent to those of ordinary skill in the art upon reading the foregoing description. Those of ordinary skill should be able to employ such variations as appropriate and the disclosure may be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein.

All references, including publications, patent applications, and patents, cited herein are hereby incorporated by reference to the same extent as if each reference were individually and specifically indicated to be incorporated by reference and were set forth in its entirety herein.

In the foregoing specification, aspects of the disclosure are described with reference to specific embodiments thereof, but those skilled in the art will recognize that the disclosure is not limited thereto. Various features and aspects of the above-described disclosure may be used individually or jointly. Further, embodiments can be utilized in any number of environments and applications beyond those described herein without departing from the broader spirit and scope of the specification. The specification and drawings are, accordingly, to be regarded as illustrative rather than restrictive.

What is claimed is:

1. A computer-implemented method comprising:
receiving data that comprises a standard resource that conforms to a Fast Healthcare Interoperability Resources (FHIR) standard, wherein the standard resource includes one or more standard data elements for representing and exchanging healthcare information;
determining that the standard resource further includes an extension that incorporates a non-standard data element into the standard resource, wherein the non-standard data element is defined by a structure definition that is external from the data;
converting the standard resource into a normalized resource that conforms with an integration model, wherein the converting includes normalizing the non-standard data element based on a schema of the integration model to generate a normalized extension element that includes an attribute that extends the standard resource, and wherein the normalized resource comprises the one or more standard data elements and the normalized extension element; and
providing the normalized resource.

2. The computer-implemented method of claim 1, wherein normalizing the non-standard data element comprises replacing one or more cells of an extension array of the extension with a concrete type data structure.

3. The computer-implemented method of claim 1, wherein determining that the standard resource further includes the extension is based on:
identifying, in the data, a reserved key that indicates an occurrence of the extension;
obtaining a uniform resource locator or address associated with the extension; and
retrieving the structure definition with the uniform resource locator or the address.

4. The computer-implemented method of claim 1, further comprising determining a name of the attribute, from the data or the structure definition, that extends the standard resource.

5. The computer-implemented method of claim 1, further comprising associating an annotation with the normalized extension element, wherein the annotation indicates that the normalized extension element is associated with the extension.

6. The computer-implemented method of claim 5, further comprising:
converting the normalized resource to a second standard resource, wherein the converting comprises:
identifying an occurrence of the annotation associated with the normalized extension element; and
converting the normalized extension element to a second non-standard extension element according to a second schema associated with the standard resource, wherein the second schema defines the standard resource and a format of the extension.

7. The computer-implemented method of claim 1, wherein providing the normalized resource comprises generating a user interface (UI) and rendering within the UI a graphical display of the normalized resource.

8. A system comprising:
one or more hardware processors; and
one or more non-transitory computer-readable media storing instructions which, when executed by the one or more hardware processors, cause the system to perform operations comprising:
determining an occurrence of a standard resource that includes one or more standard data elements and an extension that incorporates a non-standard data element within data that represents healthcare information, wherein the non-standard data element is defined by an external structure definition;
normalizing the non-standard data element based on an integration model to define a normalized extension element that includes an attribute that extends the standard resource;
converting the standard resource into a normalized resource that conforms with the integration model, wherein the normalized resource comprises the one or more standard data elements and the normalized extension element; and
performing one or more operations using the normalized resource.

9. The system of claim 8, wherein normalizing the non-standard data element comprises replacing one or more cells of an extension array of the extension with a concrete type data structure.

10. The system of claim 8, wherein determining the occurrence of the standard resource that includes the one or more standard data elements and the extension, is based on:
identifying a reserved key that indicates an occurrence of the extension;
obtaining a uniform resource locator or address associated with the extension; and
retrieving the external structure definition with the uniform resource locator or the address.

11. The system of claim 8, the operations further comprising determining a name of the attribute, from the data or the external structure definition, that extends the standard resource.

12. The system of claim 8, the operations further comprising associating an annotation with the normalized extension element, wherein the annotation indicates that the normalized extension element is associated with the extension.

13. The system of claim 12, the operations further comprising:
converting the normalized resource to a second standard resource, wherein the converting comprises:
identifying an occurrence of the annotation associated with the normalized extension element; and
converting the normalized extension element to a second non-standard extension element according to a second schema associated with the standard resource, wherein the second schema defines the standard resource and a format of the extension.

14. The system of claim 8, wherein performing the one or more operations using the normalized resource comprises generating a user interface (UI) and rendering within the UI a graphical display of the normalized resource.

15. A non-transitory computer-readable medium storing instructions which, when executed by one or more processing systems, cause operations to be performed comprising:
determining an occurrence of a standard resource that includes one or more standard data elements and an extension that incorporates a non-standard data element within data that represents healthcare information, wherein the non-standard data element is defined by an external structure definition;
normalizing the non-standard data element based on an integration model to define a normalized extension element that includes an attribute that extends the standard resource;
converting the standard resource into a normalized resource that conforms with the integration model, wherein the normalized resource comprises the one or more standard data elements and the normalized extension element; and
performing one or more operations using the normalized resource.

16. The non-transitory computer-readable medium of claim 15, wherein normalizing the non-standard data element comprises replacing one or more cells of an extension array of the extension with a concrete type data structure.

17. The non-transitory computer-readable medium of claim 15, wherein determining the occurrence of the standard resource that includes the one or more standard data elements and the extension, is based on:
identifying a reserved key that indicates an occurrence of the extension;
obtaining a uniform resource locator or address associated with the extension; and
retrieving the external structure definition with the uniform resource locator or the address.

18. The non-transitory computer-readable medium of claim 15, the operations further comprising determining a name of the attribute, from the data or the external structure definition, that extends the standard resource.

19. The non-transitory computer-readable medium of claim 15, the operations further comprising associating an annotation with the normalized extension element, wherein the annotation indicates that the normalized extension element is associated with the extension.

20. The non-transitory computer-readable medium of claim 19, the operations further comprising:
converting the normalized resource to a second standard resource, wherein the converting comprises:
identifying an occurrence of the annotation associated with the normalized extension element; and
converting the normalized extension element to a second non-standard extension element according to a second schema associated with the standard resource, wherein the second schema defines the standard resource and a format of the extension.
